(19) United States
(12) Patent Application Publication
(10) Pub. No.: US 2008/0118440 A1
(43) Pub. Date: May 22, 2008

(54) Imaging Cellular Nucleic Acids

(75) Inventors: Philip Kuocherng Liu, Winchester, MA (US); Young Ro Kim, Lynn, MA (US); Bruce R. Rosen, Lexington, MA (US); Christina Huang Liu, Winchester, MA (US)

Correspondence Address: FISH & RICHARDSON PC, P.O. BOX 1022, MINNEAPOLIS, MN, 55440-1022, US

(73) Assignee: THE GENERAL HOSPITAL CORPORATION, Boston, MA (US)

(21) Appl. No.: 11/574,160

(22) PCT Filed: Aug. 23, 2005
(86) PCT No.: PCT/US05/29875

§ 371 Date: Oct. 5, 2007

Related U.S. Application Data
(60) Provisional application No. 60/603,907, filed on Aug. 23, 2004.

(51) Int. Cl. A61K 49/00; A61P 43/00

(52) U.S. Cl. 424/9.35

(57) ABSTRACT

A reporter conjugate for non-invasive imaging of gene expression in vivo is disclosed. The conjugate includes a targeting nucleic acid linked to a contrast agent, such as a paramagnetic label that can be used with magnetic resonance imaging (MRI). The targeting nucleic acid can be an anti-sense strand that hybridizes to a portion of a messenger RNA encoded by the gene whose expression is to be imaged. In some embodiments, the contrast agent is a chelated metal such as gadolinium or dysprosium. The invention also features methods to image gene expression in various tissues, including the brain.

### CROSS-REFERENCE TO RELATED APPLICATIONS

This application claims the benefit of U.S. Provisional Application Ser. No. 60/603,907, filed on Aug. 23, 2004, the contents of which are incorporated herein by reference in their entirety.

### GOVERNMENT SUPPORT

The inventions described and claimed herein were made with government support under R01 454845, awarded by NINDS. The government has certain rights in this application.

### TECHNICAL FIELD

This invention relates to imaging cellular nucleic acids, such as imaging the delivery, uptake, and/or expression of a nucleic acid within cells in various tissues using, e.g., magnetic resonance imaging (MRI), and more particularly to MR imaging of gene expression in the brain.

### BACKGROUND

A number of different approaches to imaging cells have been investigated using either optical, e.g., using green fluorescent protein, bioluminescence, or near infrared fluorescence, or nuclear imaging techniques. Common limitations to these techniques are limited penetration depth (optical techniques) or spatial resolution (nuclear techniques). Recent advances in magnetic resonance (MR) imaging and in particular MR microscopy have led to improved image resolution. However, compared to optical and nuclear techniques, molecular probe detection by MR is several magnitudes less sensitive. On the other hand, MR imaging offers much improved spatial resolution with anatomical precision compared to other modalities such as optical imaging, computer tomography (CT), and positron emission tomography (PET).

In all of these imaging modalities, the common goal is to deliver a suitable contrast agent or label to the relevant tissue, and more specifically into the cells. In the brain, for example, one must typically find a way to overcome the blood-brain-barrier. In addition, most of the known contrast agents, for example, for MRI, have limited permeability to cells when administered to live subjects, and as a result the limited permeability provides only a short and often unstable window for MR imaging.

### SUMMARY

The invention is based, in part, on the discovery that short nucleic acid sequences, e.g., phosphorothioated nucleic acid sequences, can be linked to one or more reporter groups to form reporter conjugates, which transport the reporter groups into cells, to cell membranes, or into the vicinity of cells in which a specific cellular nucleic acid, e.g., an RNA, DNA, gene, or chromosome, without the need for translocation sequences or the like. Liposomes may aid the uptake of the reporter conjugates into cells. By properly designing the nucleic acid to target a specific nucleic acid sequence in a cell, such as a messenger RNA transcribed from a target gene, the new reporter conjugates can be used to image expression of target cellular nucleic acids non-invasively in a variety of tissues, such as tissues of the brain, liver, pancreas, heart, lung, spinal cord, prostate, breast, gastrointestinal tract, ovary, and kidney.

The reporter group can be an MRI contrast agent, such as a paramagnetic label, such as a superparamagnetic iron oxide particle whose maximum diameter is between 1 nm and 2000 nm, e.g., between 2 nm and 1000 nm. In some embodiments, the maximum particle diameter is between 10 nm and 100 nm. The particle can be attached to the targeting nucleic acid through entrapment in a cross-linked dextran.

In some embodiments, the paramagnetic label is a chelated metal such as Gd³⁺ or Dy³⁺. The reporter group can also be a fluorescent label, e.g., FITCs, Texas Red, or Rhodamine.

In one aspect, the invention features reporter conjugates for imaging cellular nucleic acids that include a single targeting nucleic acid linked to one or more reporter groups. These targeting nucleic acids and reporter groups are described in detail herein.

In another aspect, the invention features methods of imaging a cellular nucleic acid in a tissue in vivo. The methods include obtaining a reporter conjugate including a targeting nucleic acid linked to a reporter group, wherein the targeting nucleic acid hybridizes to a target nucleic acid molecule corresponding to the cellular nucleic acid to be imaged; administering the reporter conjugate to the tissue in an amount sufficient to provide a detectable image; allowing sufficient time to pass to allow any unbound reporter conjugate to leave the tissue; and imaging the tissue, wherein a detectable image of the reporter group in the tissue indicates the presence of the cellular nucleic acid. The target nucleic acid molecule can include a messenger RNA transcribed from a target gene, and the targeting nucleic acid can include an anti-sense strand that hybridizes to a portion of the messenger RNA, wherein the presence of the cellular nucleic acid indicates expression of the target gene. The target gene can be a therapeutic gene previously delivered to the tissue. The tissue can be, e.g., brain, heart, lung, liver, pancreas, spinal cord, prostate, breast, gastrointestinal system, ovary, or kidney tissue. The tissue can be in a patient, e.g., a human patient. The reporter group can be a superparamagnetic iron oxide particle whose maximum diameter is between 1 nm and 2000 nm. The reporter conjugate can be administered by, e.g., intravenous injection or intra-cerebroventricular infusion.

In another aspect, the invention features reporter conjugates for imaging cellular nucleic acids that include a single targeting nucleic acid linked to one or more superparamagnetic iron oxide particles whose maximum diameter is between 1 nm and 1000 nm (e.g., between 10 and 100 mm). In some embodiments, the particles are a monocrystalline iron oxide nanoparticle (MION), ultra small superparamagnetic iron oxide particle (USPIO), or cross-linked iron oxide (CLIO) particle. The particle can be surrounded by cross-linked dextran.

In another aspect, the invention features methods of imaging target cells (e.g., cornu ammonis neurons) that are undergoing or have undergone programmed cell death in a tissue. The methods include obtaining a reporter conjugate including a targeting nucleic acid linked to a reporter group, wherein the targeting nucleic acid hybridizes to a target nucleic acid molecule corresponding to the target cells; administering the reporter conjugate to the tissue in an amount sufficient to provide a detectable image; allowing sufficient time to pass to allow any unbound reporter conjugate to leave the tissue; and imaging the tissue, wherein a presence of a detectable image of the reporter group in the tissue indicates that the cells in the tissue have not undergone programmed cell death, and an absence of a detectable image of the reporter group indicates that the cells are undergoing or have undergone programmed cell death.

In another aspect, the invention features methods of treating a disorder, e.g., a cancer, in a patient. The methods include obtaining a conjugate including a targeting nucleic acid linked to a therapeutic agent and a reporter group, wherein the targeting nucleic acid hybridizes to a target nucleic acid molecule corresponding to a target organ or tissue; and administering the conjugate to a patient in an amount sufficient to treat the disorder. In some embodiments, the targeting nucleic acid preferentially binds to an oncogene or a mutant mRNA transcribed by an oncogene.

In another aspect, the invention features the use of a reporter conjugate including a targeting nucleic acid linked to a reporter group, wherein the targeting nucleic acid hybridizes to a target nucleic acid molecule corresponding a cellular nucleic acid, in the preparation of a pharmaceutical composition for imaging a cellular nucleic acid in a tissue in vivo

In another aspect, the invention features methods of imaging expression of a target gene in a tissue in vivo, by obtaining a reporter conjugate including a targeting nucleic acid linked to a reporter group, wherein the targeting nucleic acid hybridizes to a target nucleic acid molecule corresponding to the target gene whose expression is to be imaged; administering the reporter conjugate to the tissue in an amount sufficient to provide a detectable image; allowing sufficient time to pass to allow any unbound reporter conjugate to leave the tissue; and imaging the tissue, wherein a detectable image of the reporter group in the tissue indicates that the target gene has been expressed.

In other aspects, the invention features methods of imaging a cellular nucleic acid in a tissue by obtaining a reporter conjugate including a targeting nucleic acid linked to a reporter group, wherein the targeting nucleic acid hybridizes to a target nucleic acid molecule corresponding to the cellular nucleic acid to be imaged; administering the reporter conjugate to the tissue in an amount sufficient to provide a detectable image; allowing sufficient time to pass to allow any unbound reporter conjugate to leave the tissue; and imaging the tissue, wherein a detectable image of the reporter group in the tissue indicates the presence of the cellular nucleic acid.

The invention also includes methods of treating a cancer cell in a patient by obtaining a conjugate including a targeting-nucleic acid linked to an anti-cancer agent, wherein the targeting nucleic acid hybridizes to a target nucleic acid molecule corresponding to the cancer cell; and administering the conjugate to the patient in an amount sufficient to inhibit growth of the cancer cell. The conjugate can further include a reporter group.

The invention also includes methods of treating a disorder in a patient by obtaining a conjugate including a targeting nucleic acid linked to a therapeutic agent, e.g., a dextran-coated therapeutic agent, wherein the targeting nucleic acid hybridizes to a target nucleic acid molecule corresponding to a desired target organ or tissue, and administering the conjugate to the patient in an amount sufficient to treat the disorder. The conjugate can further include a reporter group.

In other embodiments, the invention includes methods of decreasing expression of a gene in a cell and, optionally, imaging a cellular nucleic acid by obtaining a reporter conjugate including a nucleic acid, e.g., a phosphorothioated nucleic acid (e.g., a phosphorothioated RNA), that decreases (e.g., is designed to decrease) expression of a target gene, and administering the conjugate to a cell in an amount sufficient to decrease expression of the target gene, and, optionally, allowing sufficient time to pass to allow any unbound reporter conjugate to leave the tissue; and imaging the tissue. The nucleic acid can be, e.g., an antisense nucleic acid, a short inhibitory RNA (siRNA), a micro RNA (miRNA), or a double stranded RNA (dsRNA).

The invention also includes methods of imaging (e.g., visualizing or locating) a cell type that expresses a gene in a subject. The methods include obtaining a conjugate including a targeting nucleic acid linked to a reporter group, wherein the targeting nucleic acid hybridizes to a target nucleic acid molecule that is expressed by the cell type to be imaged, administering the conjugate to a subject in an amount sufficient to produce a detectable image, and imaging the tissue, wherein the presence of the conjugate is indicative of the cell type. The cell type to be imaged can be, e.g., a cancer cell, a transgenic cell, or a stem cell (e.g., an embryonic stem cell).

In another embodiment, the invention includes use of a reporter conjugate including a targeting nucleic acid linked to a reporter group, wherein the targeting nucleic acid hybridizes to a target nucleic acid molecule corresponding to a cellular nucleic acid, in the preparation of a pharrmaceutical composition for imaging a cellular nucleic acid in a tissue in vivo. The reporter conjugate can further include a therapeutic agent.

A nucleic acid that hybridizes or binds “specifically” to a target nucleic acid hybridizes or binds preferentially to the target, and does not substantially bind to other molecules or compounds in a biological sample.

As used herein, “paramagnetic” means having positive magnetic susceptibility and lacking magnetic hysteresis (ferromagnetism).

As used herein, an “oncogene” is an allele of a gene that is associated with increased risk of cancer, e.g., a mutant form of a proto-oncogene or tumor suppressor gene, or a viral oncogene. Numerous examples of oncogenes are known in the art (see, e.g., Vogelstein and Kinzler, Nat. Med., 10:789-99 (2004))

The new conjugates and methods allow real time imaging, such as MRI, and avoid the need for biopsies. The imaging is safe and can be performed as often as is needed for several days.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1H are a series of schematic representations of reporter conjugates, showing certain possible attachments of reporter groups, such as contrast agents or labels, that can be linked, e.g., via covalent bonds, directly or indirectly to the 5′ (FIGS. 1A and 1C) or 3′ terminus (FIGS. 1B and 1D), individually (FIGS. 1A to 1D) or at both ends (FIGS. 1E to 1H), or with additional sites within the targeting nucleic acids. Reporter groups, e.g., contrast agents and labels that can be used include, but are not limited to, paramagnetic agents, fluorescent labels (FITC, rhodamine, Texas Red), radioactive isotopes, individually or combinations. More than 50 unique reporter groups can be made in an average length (2 kilobases) of a gene transcript (mRNA). For example, 50 different reporter conjugates can be made that specifically bind to a specific target nucleic acid, e.g., to different portions of the same target. All 50 conjugates can have the same or different reporter groups, and could have different (e.g., up to 50 different) reporter groups on the 50 different conjugates. This can be used to provide signal amplification. In addition, similar numbers of reporter contrast agents can be made to the exons of a given gene.

FIG. 11 is a legend depicting the symbols used in FIGS. 1A to 1H.

FIG. 2 is an ex vivo MR image (3D GE, TR/TE=100/21 ms) showing signal reduction in various brain structures (the cortex, corpus callosum (CC), comu Ammonis (CA), and striatum) and infusion site (*) two days after MION-A26 (SEQ ID NO:1) infusion (Fe=1.4 μg). The signal is indicative of MION presence.

FIGS. 3A to 3C are micrographs that show the presence of iron oxide (MION stained with Prussian blue and tissue was counterstained for nuclei using Nuclear Fast Red) in cells at the same regions of different animals shown in the MRI (FIG. 2) 11 days after infusion of the reporter MION-A26 (SEQ ID NO:1). FIGS. 3B and 3C are inserts from FIG. 3A that show that iron oxide was present around and within the nuclei in the cortex (3B) and CA neurons (3C). FIG. 3A shows the corpus callosum. Arrows show the presence of iron oxide in perivascular spaces that connect the cerebral ventricular space to brain cells. The bar in each micrograph indicates 20 micrometer (μm).

FIG. 4 is a fluorescence micrograph that shows the distribution of fluorescent-ODN in the brain (as condensed bright dots) after infusion of the reporter conjugate MION-A26 (SEQ ID NO:1)-digoxigenin (detected using fluorescein isothiocyanate [FITC] labeled IgG against digoxigenin). The distribution of A26 (SEQ ID NO:1) is present throughout the brain and tissue sample from −1.4 mm to the Bregma. The A26 used in this study contains a sequence that is complementary to c-fos mRNA and has been able to detect c-fos mRNA expression in the brain using in situ hybridization (see below in FIG. 5) and in situ reverse transcription polymerase chain reaction (see below in FIG. 6). The scale bar depicts 120 μm; cc=corpus callosum; CA=cornu ammonis.

FIG. 5 is an autoradiograph that shows three consecutive samples of c-fos mRNA detected using conventional assay in situ hybridization (radioactive antisense A26-³⁵S) in a postmortem mouse brain.

FIG. 6 is a micrograph of c-fos mRNA in mouse hippocampus using molecular biological assay in another postmortem mouse brain by using the techniques of in situ reverse transcription PCR with and A26 labeled with digoxigenin, followed by detection using alkaline phosphatase-labeled antibody against digoxigenin.

FIG. 7 shows a serial T2* map of mouse brain that received a MION-s-ODN reporter conjugate immediately (<30 minutes; top row) and one day (bottom row) post infusion from the same subject. T2* maps of contiguous 0.5 mm MR slices from selected posterior to anterior portion of the brain (five contiguous slices, −1.5 to −3.5 mm to the bregma) are shown. The presence of MION results in regions of signal reduction (therefore, decreased T2*, arrows) that are apparent one day after infusion.

FIGS. 8A and 8B, and FIGS. 9A and 9B are representative MR images (GE, TR/TE=500/2.3 milliseconds) from 4 live mice at two or three time points (one panel per animal shown in raustral view of five contiguous brain slice images). The persistent signal reduction within the ventricles, indicative of MION presence, can still be observed (FIG. 8A) at day 1 after infusion of MION-A26 (SEQ ID NO:1). FIG. 8B shows no retention of MION in live animals that received a mixture of unconjugated MION and s-ODN. The MION signal dissipated within three hours.

FIGS. 9A to 9B shows MR images of animals, 30 minutes and 3 hours after infusion of MION-dextran (9A) (a control that shows no retention) or MION-dUTP (9B) (another control that also shows no retention).

FIGS. 10A to 10C are bar graphs (10A and 10B) and MR images of mouse brain (10C). FIGS. 10A and 10B compare R2*(1/T2*) values in contralateral cortical regions (boxes in FIG. 10C) from selected brain slices of mice injected with MION-A26 (SEQ ID NO:1) and MION immediately (<30 minutes, 10A) or 1 day after infusion (10B). The bar graph shows that the brain cells retained significant amounts of the MION reporter conjugate, and that it was distributed from the left ventricle (infusion site) to the right ventricle and the cortex.

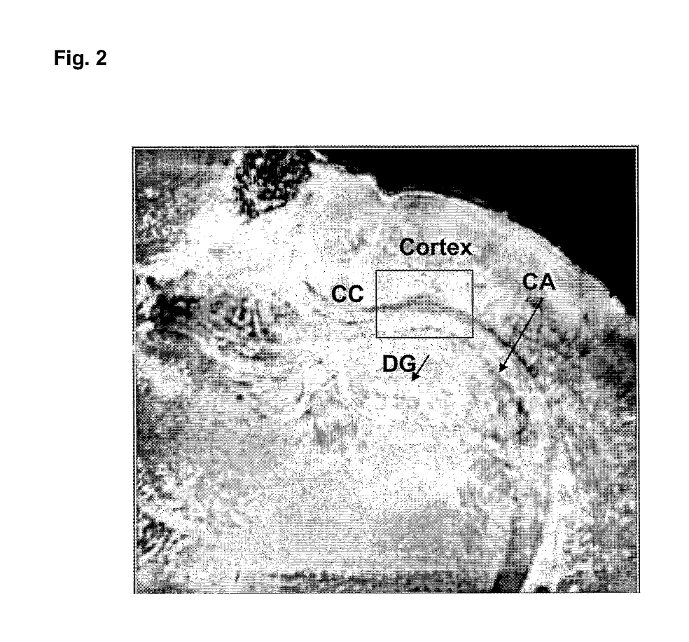

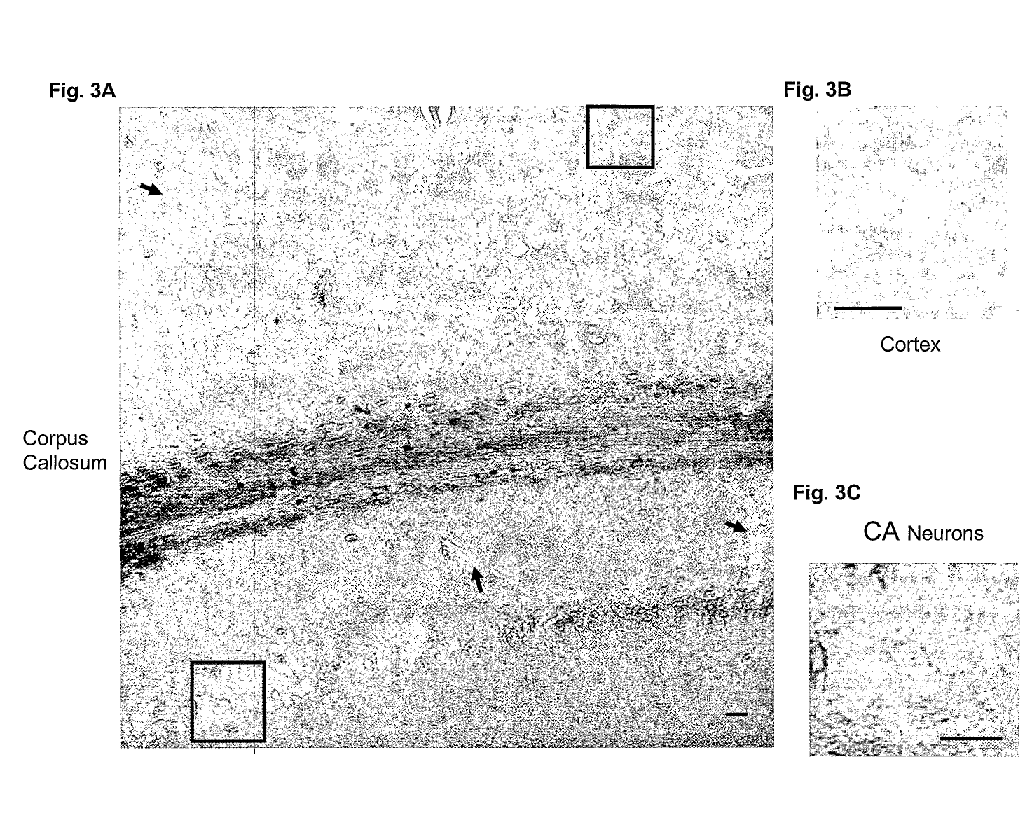

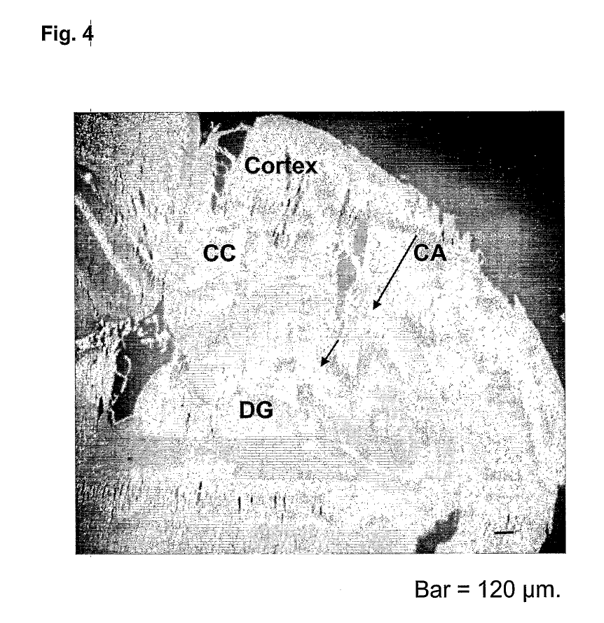

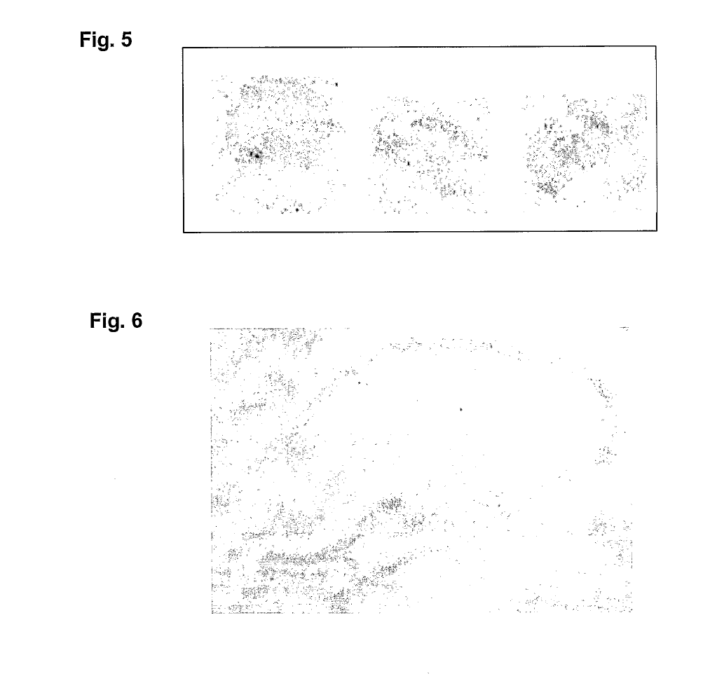

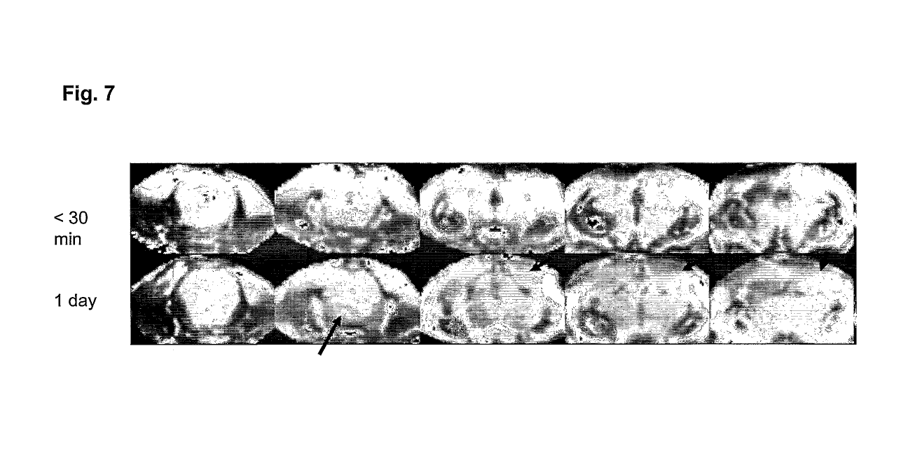

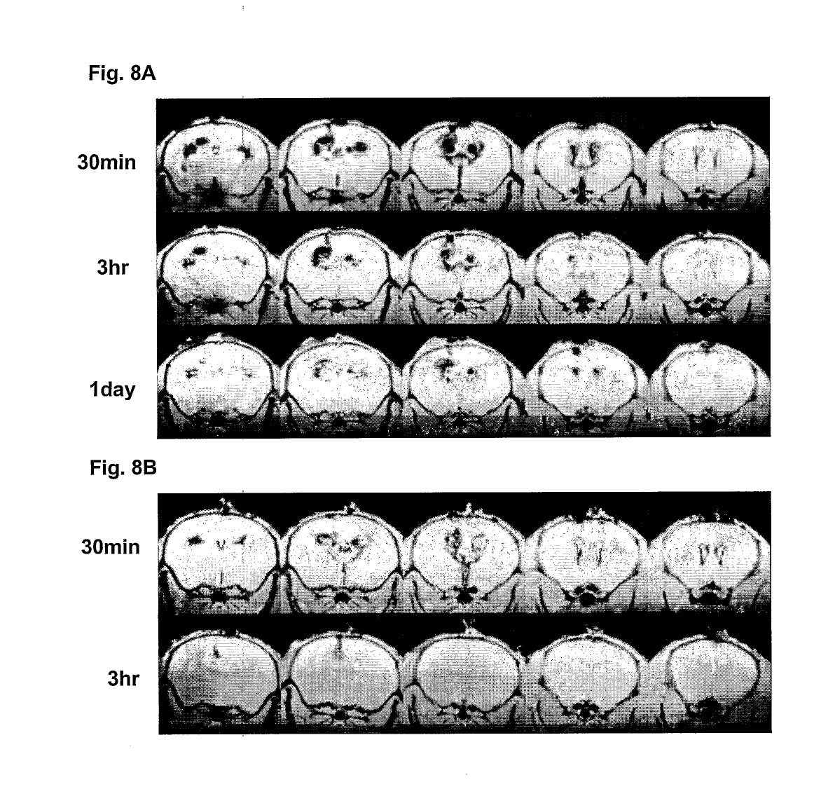

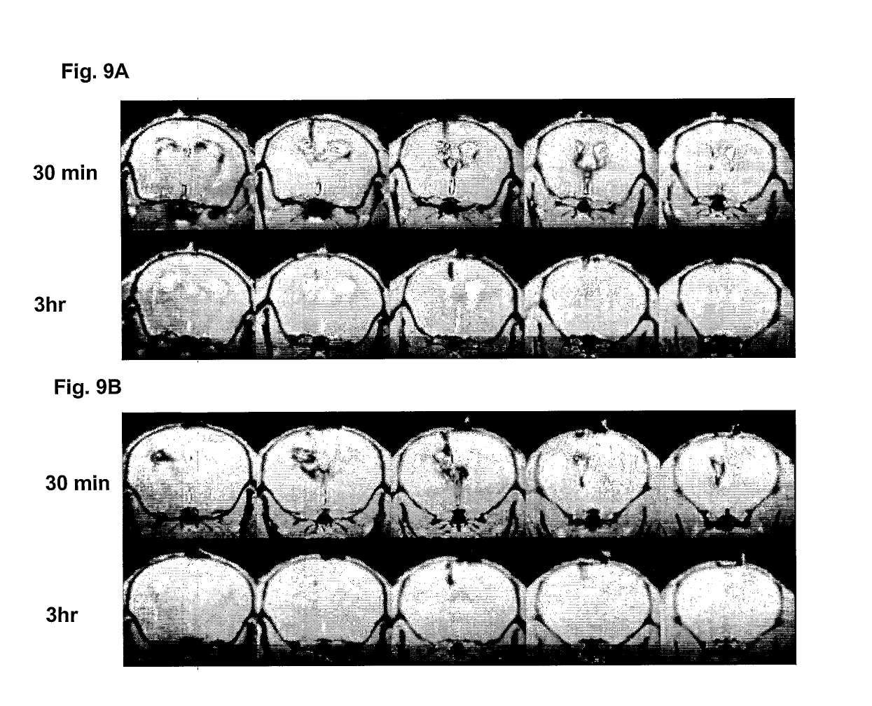

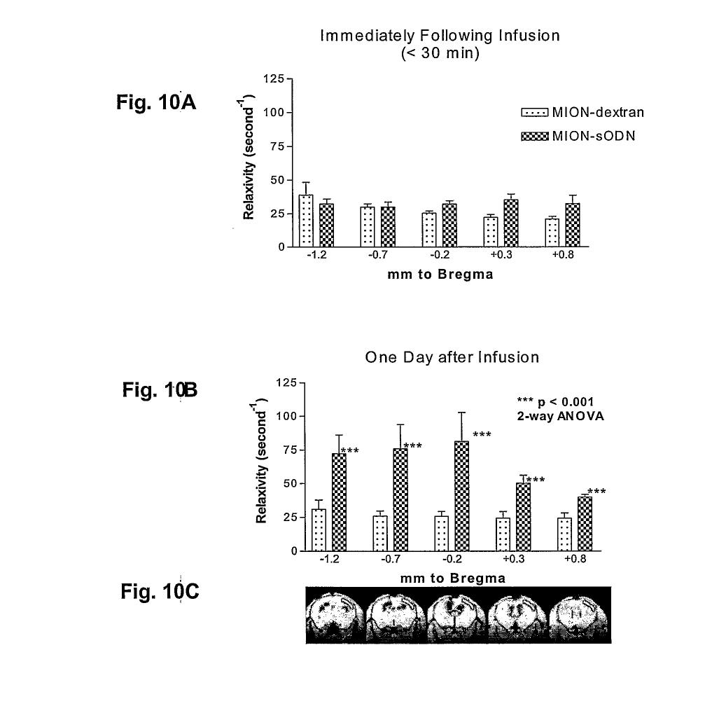

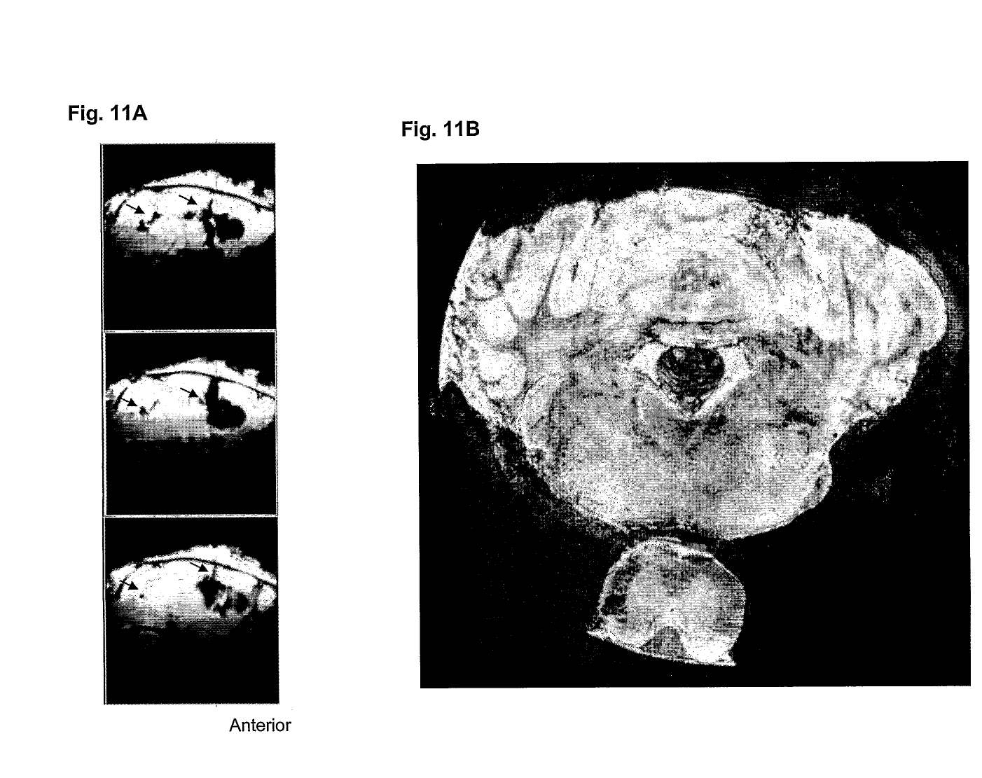

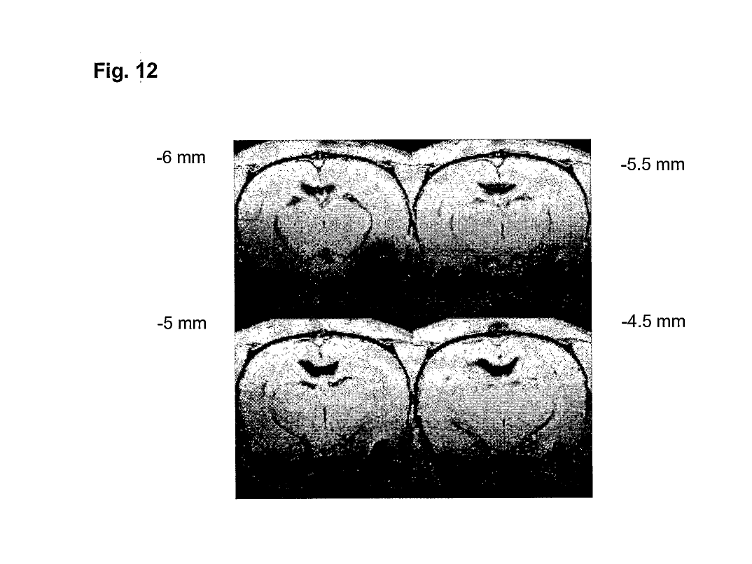

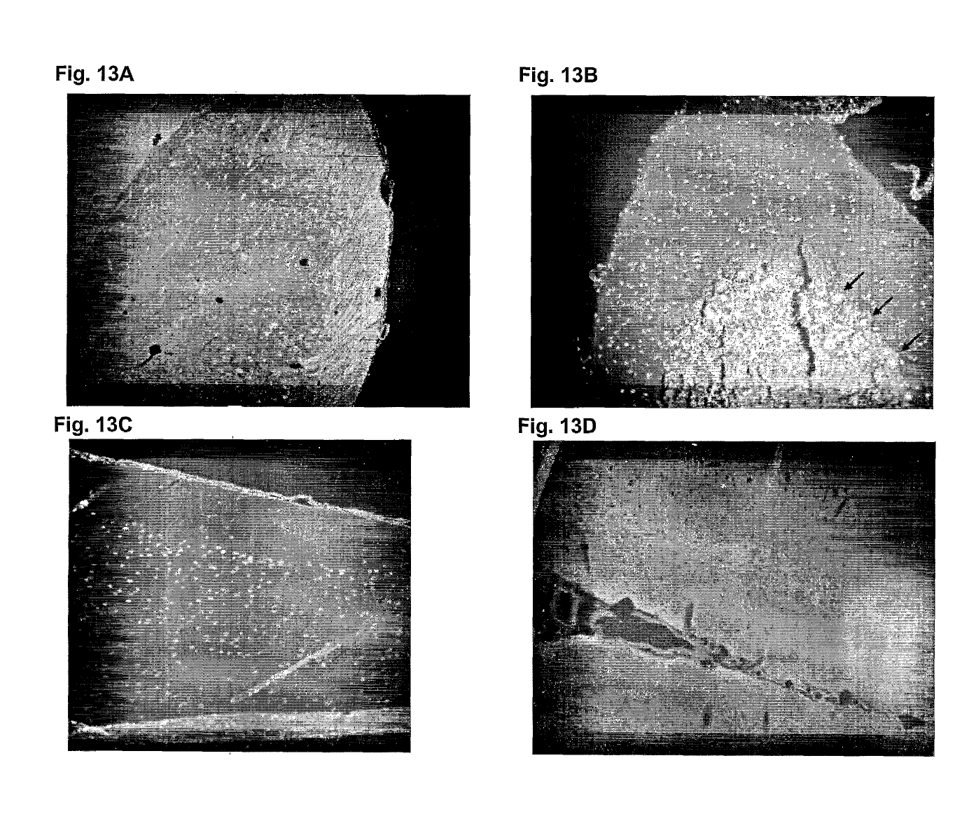

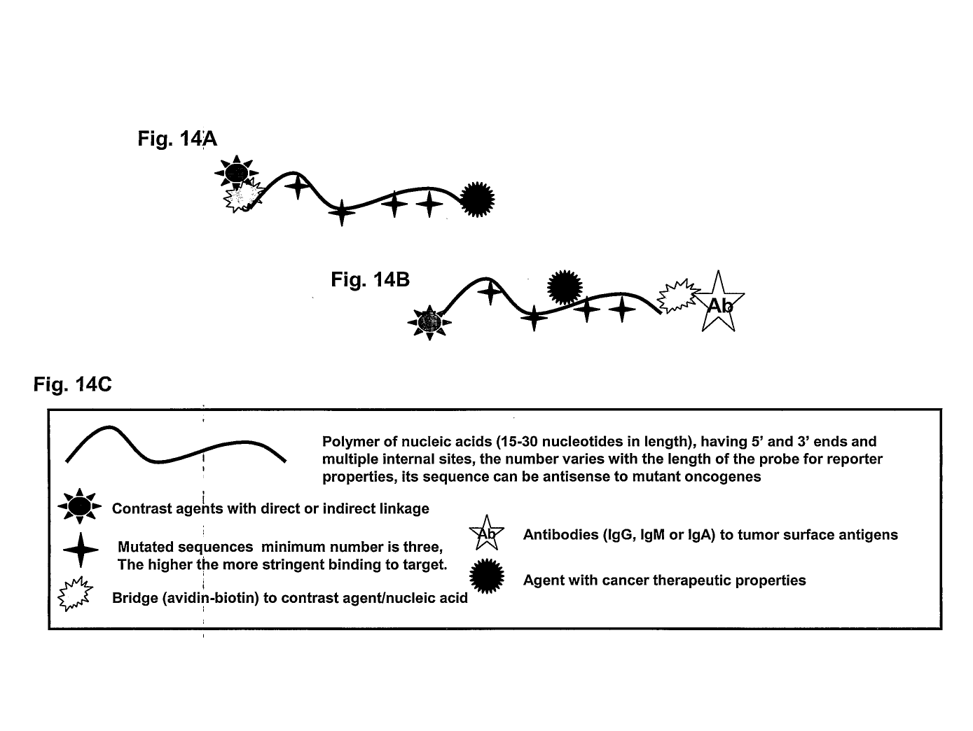

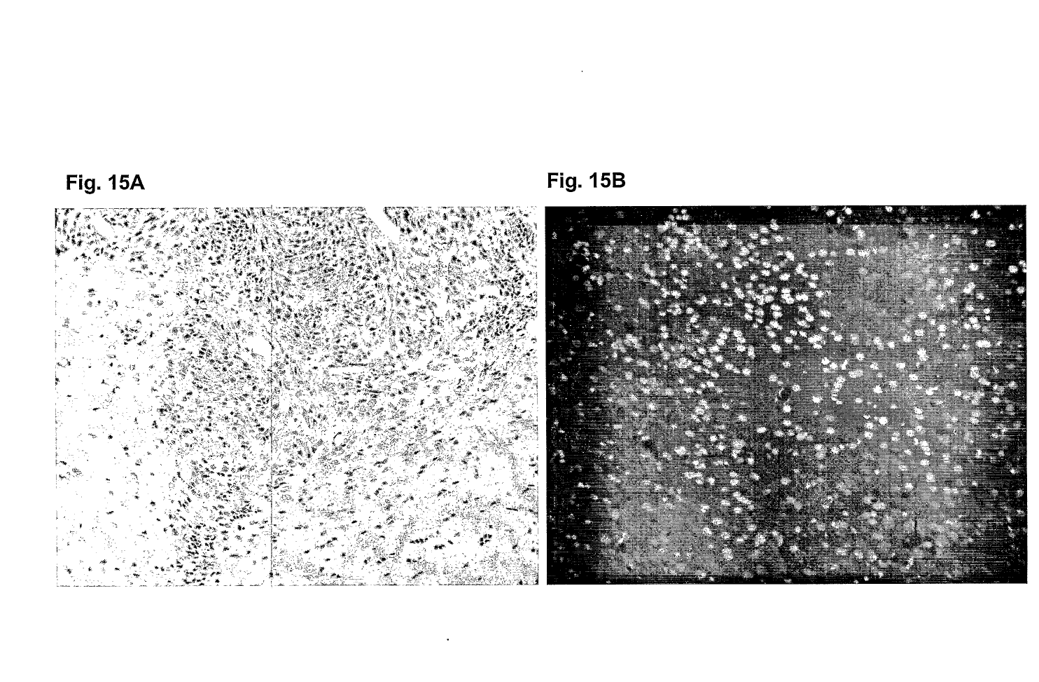

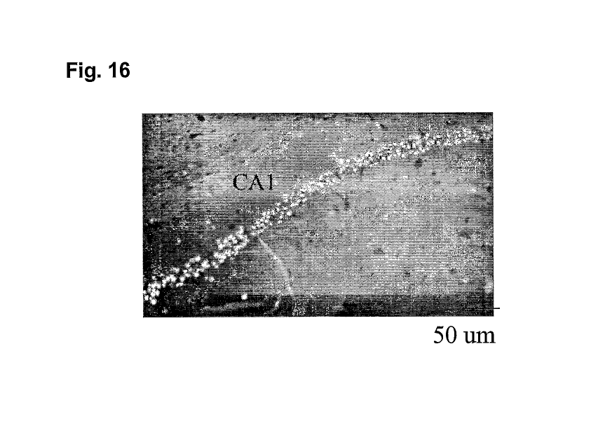

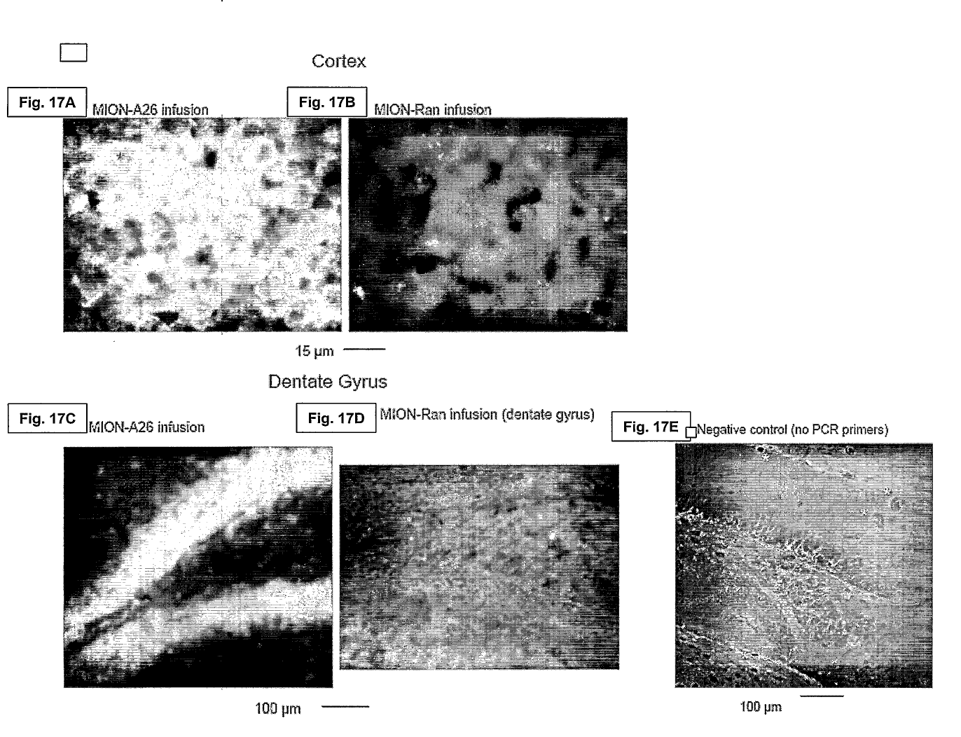

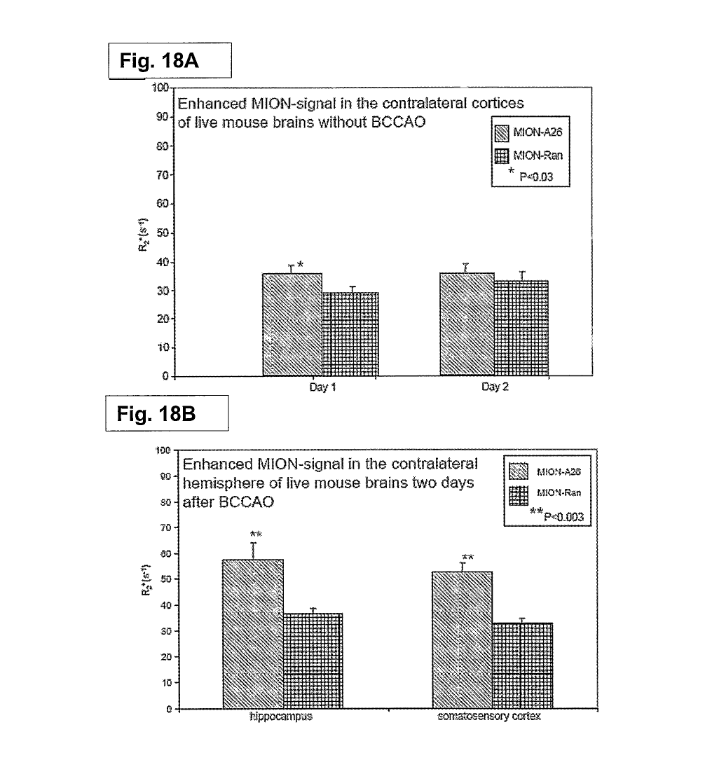

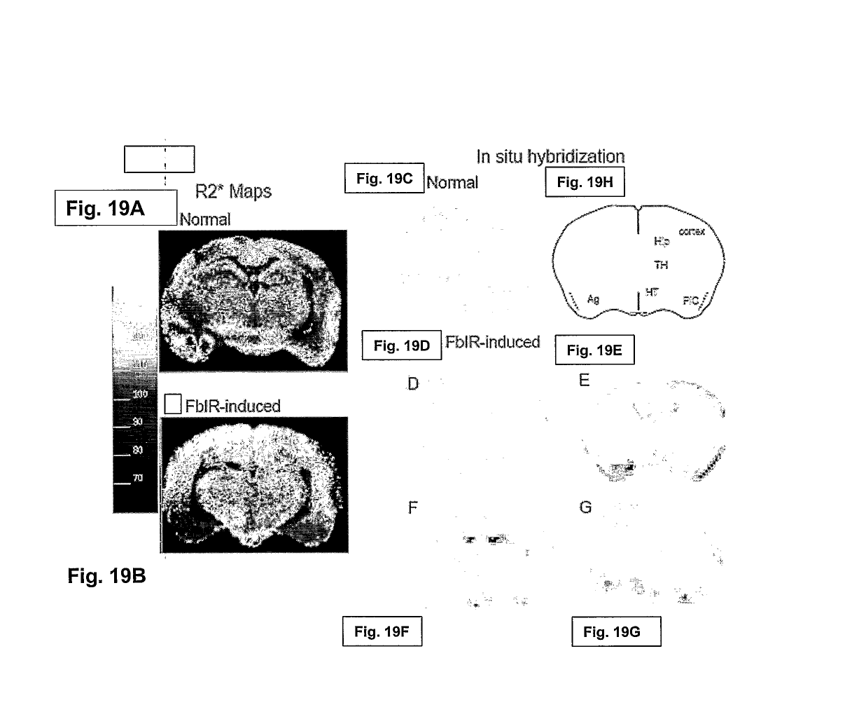

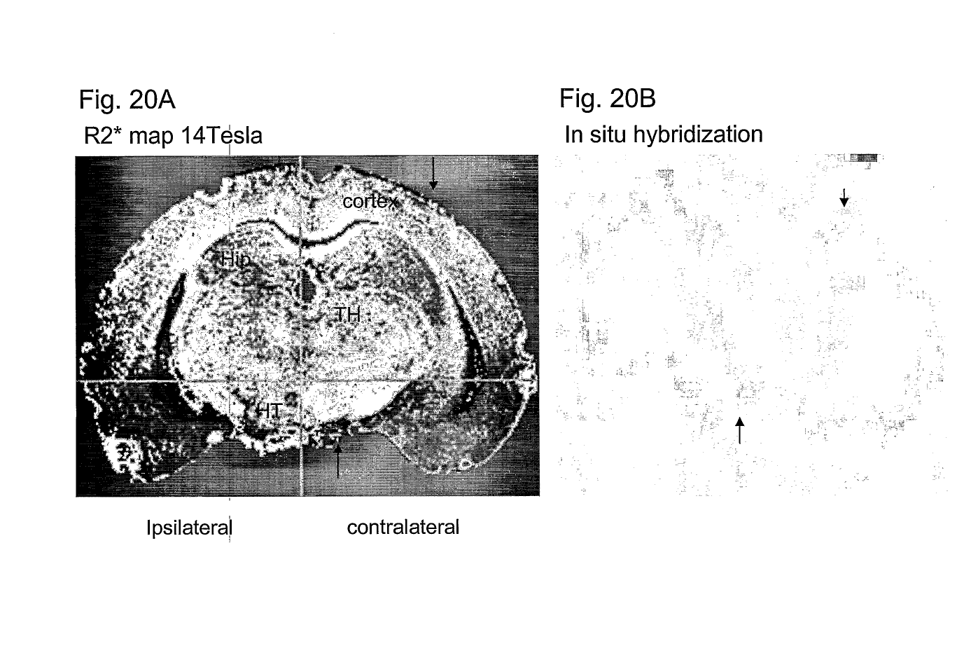

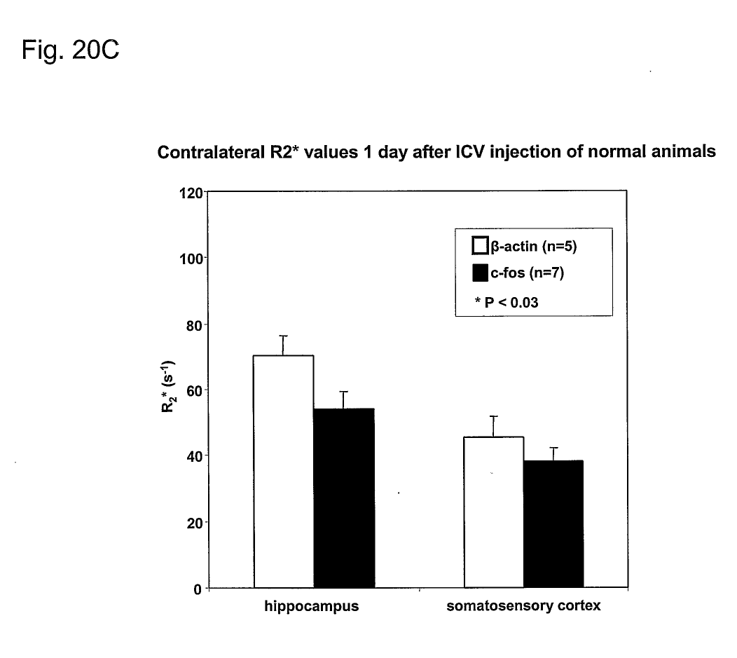

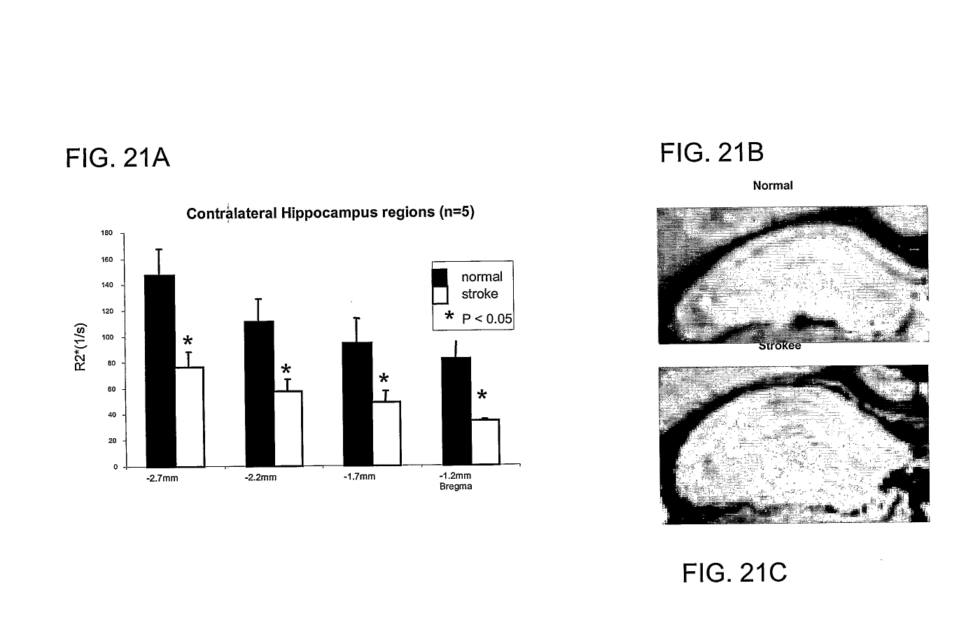

FIGS. 11A and 11B are MR images of an animal brain that received MION-A26(SEQ ID NO:1). The images were acquired at three days after the infusion. The distribution of MION can be seen in the cerebellum (FIG. 11A; in vivo MRI, sagittal view) and in the white matter of the spinal cord (FIG. 11B, ex vivo MRI).

FIG. 12 is a high resolution T2-weighted MR brain image of a live rat acquired at 4.7 Tesla (GE, TR/TE=1000/8 ms) at −4.5 to −6 mm to the bregma, 24 hours after reporter MION-A26 (SEQ ID NO:1) infusion. The image shows a homogenous retention of reporter MION-A26 (SEQ ID NO:1). Signal reduction can be observed in areas such as the cortex, white matter tracks, hippocampus and the striatum.

FIGS. 13A to 13D are a series of fluorescence micrographs of a rat brain that show the presence of s-ODN (from an infusion of the reporter conjugate MION-A26 (SEQ ID NO:1)-digoxigenin) as clustered bright dots in the olfactory lobe (13A), the cerebellum (13B) and the cortex (13C). FIG. 13D is a control that shows background fluorescence in a sample without a fluorescent antibody label. The arrows in FIG. 13B denote Purkinje cells.

FIGS. 14A and 14B are two schematic representations of reporter conjugates that can be used for therapeutic purposes, e.g., to treat cancer, when a target gene contains a known mutation.

FIG. 14C is a legend depicting the symbols used in FIGS. 14A and 14B.

FIGS. 15A and 15B are micrographs that show a preferential distribution of digoxigenin-labeled A26 (anti-sense to c-fos mRNA) in glioblastoma in rats. Cells of the glioblastoma cell line D74 (ENU-induced, 105 each rat) were implanted to the cortex of a Fisher 344 rat. One week later, digoxigenin labeled s-ODN (1 nmol) was infused via ICV route on the contralateral hemisphere. One day after infusion, the reporter conjugate remains in the glioblastoma cells as indicated by HE staining (15A) and by fluorescence of FITC-labeled antibodies to digoxigenin (15B).

FIG. 16 is a micrograph of animal brain showing early programmed cell death (apoptosis) using TUNEL (terminal UTP nick end labeling) staining in the CA neurons of a mouse one day after stroke.

FIGS. 17A to 17D are fluorescent micrographs of in situ RT-PCT on postmortem mouse brain samples. In FIGS. 17A, 17C, and 17E, MION-A26 (SEQ ID NO:1) was used for reverse transcription. In FIGS. 17B and 17D, MION-Ran (SEQ ID NO:3) was used for reverse transcription. FIG. 17E depicts the negative control without PCR primers.

FIGS. 18A and 18B are bar graphs depicting R2* values of brain regions in vivo. FIG. 18A depicts MION signal in (as elevated R2*) the contralateral cortices on days 1 and 2 following infusion with MION-A26 or MION-Ran. FIG. 18B depicts MION signal in the contralateral hemisphere two days after global cerebral ischemia induced using bilateral common carotid arteries occlusion (BCCAO) and infusion with MION-A26 or MION-Ran.

FIGS. 19A and 19B are c-fos mRNA expression maps of mouse brains produced by R2* map computed from ex vivo MR microscopy. Brightness indicates increased signal intensity.

FIGS. 19C to 19G are c-fos mRNA expression maps of mouse brains produced by in situ hybridization. In situ hybridization was performed on mice without BCCAO (19C) and 30, 60, 120, and 240 minutes following a transient 30-minute BCCAO and reperfusion (19D-19G).

FIG. 19H is a schematic map of C57b16 mouse showing brain regions where elevated c-fos mRNA is expressed (Paxinos and Franklin, The Mouse Brain in Stereotaxic Coordinates, Academic Press Limited, London, 2001). Ag=amygdala, TH=thalamus, HT=hypothalamus, Hip=hippocampus, Pic=piriform cortex.

FIG. 20A is a beta-actin mRNA map of mouse brains produced by R2* map (acquired similar to FIGS. 19A and 19B). Brightness indicates increased signal intensity. TH=thalamus, HT=hypothalamus, Hip=hippocampus.

FIG. 20B is a c-fos mRNA expression map of mouse brain produced by in situ hybridization. In situ hybridization was performed on mice without BCCAO.

FIG. 20C is a bar graph depicting R2* values of brain regions of normal animals in vivo one day following infusion with MION-A26 (c-fos) or MION-BA25A1 (ACGCAGCTCAGTAACAGTCCGCCTA 0(SEQ ID NO:6); Alonso et al., J. Mol. Evol., 23:11-12, 1986).

FIG. 21A is a bar graph depicting R2* from hippocampus in live animals with or without BCCAO and infused with MION-ODN.

FIGS. 21B and 21C are MR images of MION-ODN retention in the hippocampus of normal (21B) or stroke model (21C) mice.

### DETAILED DESCRIPTION

The invention relates to new methods and compositions for imaging the uptake/distribution and/or expression of specific target genes, such as therapeutic genes, in various cells and tissues, such as in the brain, non-invasively using various imaging modalities, such as MRI.

The general methodology and reporter conjugates, as well as applications for the new reporter conjugates will be described, and the examples will show that the reporter conjugates (MION-s-ODN), after delivery to live subjects, can be internalized by brain cells (FIGS. 2-3); the cells that have retained the MION and ODN (FIG. 4) express c-fos messenger RNA, and the labeled antisense ODN can detect its expression (FIGS. 5-6). Then the examples will show that the reporter conjugate MION-s-ODN can be used to produce MR images of the MION and ODN in the brain in live subjects (FIGS. 7-12).

### General Methodology

The new imaging methods use novel reporter conjugates to image the uptake and distribution of targeting nucleic acids, e.g., oligodeoxyribonucleotides (ODN), delivered to the brain or other tissues in live animals and humans. The conjugates include a reporter group, such as a contrast agent or a label, e.g., an MRI contrast agent, e.g., iron oxide nanoparticles (e.g., MION-dextran) linked to a targeting nucleic acid (such as a single-stranded ODN) that hybridizes to a portion of a particular target nucleic acid molecule. The conjugate is delivered to the tissue containing, or thought to contain, a target gene, whose uptake, distribution, or expression is to be imaged. For example, if the reporter conjugate is to be delivered to the brain, one can use convection-enhanced delivery to the cerebral ventricles such as to the lateral ventricle (Liu et al., 1994 and Cui et al., 1999) or the 4^{th} ventricles (Sandberg et al., J. Neuro-Oncology, 58:187-192, 2002). Delivery can also be intrathecal (Liu et al. (2004) Magn. Reson. Med. 51:978-87) or by any additional routes that lead directly or indirectly to brain cells.

After a sufficient amount of time for the reporter conjugate to be localized to, and internalized by, the appropriate cells within the tissue, and for any unbound conjugates to leave the tissue, the tissue is imaged. For example, the tissue can be imaged with a series of high-resolution T2*-weighted MR images, e.g., taken 1, 2, or 3 days after infusion of the reporter conjugate.

To use the new conjugates and methods to image gene expression, the targeting nucleic acid can be prepared as an anti-sense strand that is designed to hybridize to a portion of a target messenger RNA transcribed from the target gene. Thus, if a reporter conjugate including this anti-sense strand is detected in cells in a tissue, it provides a clear indication that that target mRNA is present in the cell, and thus that the target gene is being expressed. Guidance on designing nucleic acids that hybridize to a target under specific conditions (e.g., intracellular conditions) can be found, e.g., in Ausubel et al., eds. Current Protocols in Molecular Biology, John Wiley & Sons, N.Y. (1989).

In the examples described below, immunohistochemical analysis showed that ODN were present in the cortex, hippocampus, and the corpus callosum, where iron oxide was also present. In the animals receiving no infusion or only an MION infusion (no conjugate), no apparent iron staining was detected in the brain slices. These results indicated that regions of s-ODN uptake coincided with areas of signal reduction (therefore, the contrast agent, e.g., MION, was retained by the cells).

The new reporter conjugates and imaging methods open a new venue to detect and track the delivery and uptake of nucleic acid molecules in live animals for neuroscience research and various clinical applications.

### Reporter Conjugates

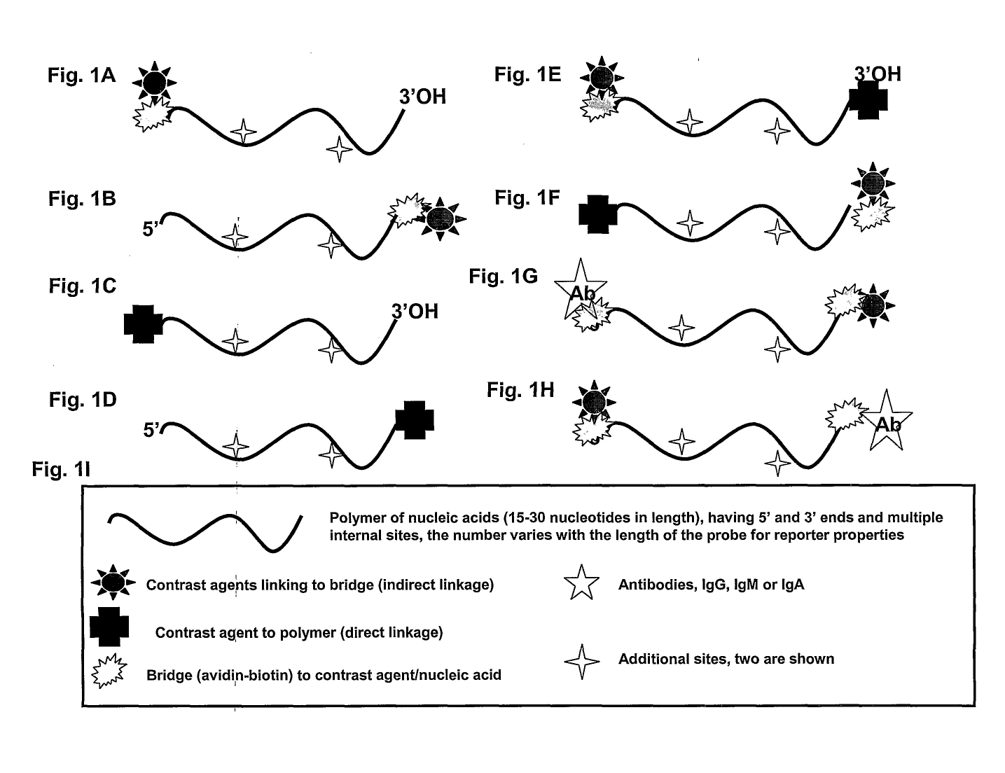

The reporter conjugates are prepared by conjugating or linking one or more targeting nucleic acids to one or more reporter groups, such as magnetic particles that change the relaxivity of the cells once internalized so that they can be imaged using NMR. One targeting nucleic acid can have multiple (e.g., 2, 3, or more) reporter groups attached (all or some the same or different), or a set of numerous reporter conjugates can be created in which they all have the same targeting nucleic acid and 2 or more different reporter groups within the set. Alternatively, a set of reporter conjugates can be made that have different targeting nucleic acids that all target different portions of the same target gene (or that target different target genes), and each have the same or different reporter groups. Several variations of the different types of reporter conjugates are shown in FIGS. 1A to 1I.

As shown in FIGS. 1A and 1B, the conjugate includes a targeting nucleic acid of 15 to 30 nucleotides (also referred to herein as an oligonucleotide or ODN), one or more reporter groups, such as a contrast agent, linked to either the 5′ or 3′ ends of the ODN, either directly, e.g., by a covalent bond or via an optional linker group or “bridge” (e.g., a linkage of a desired length) between the ODN and the reporter group(s). The targeting nucleic acid must have at least 80% sequence homology (identity) with a sequence that is complementary to a portion of the target nucleic acid molecule. For example, at least 15 nucleotides in the ODN would be complementary to a portion of the target nucleic acid, and thus will hybridize preferentially to the target nucleic acid. The targeting nucleic acid can be either single-stranded DNA or RNA, and is typically an anti-sense strand, and thus complementary, to a portion of the target nucleic acid. The ODN may include one or multiple internal sites that can be attached to a reporter group, e.g., labeled, for example, with a radioactive or fluorescent label.

FIGS. 1E to 1H show reporter conjugates that include two or more reporter groups, as well as an optional antibody that can be attached at either end of the molecule (FIGS. 1G and 1H). These antibodies are typically ones that bind specifically to cell-surface antigens of particular cells or cell types to direct the reporter conjugate to the appropriate cells. Once on the surface of the cell, the reporter conjugates pass through the cell membrane and into the cells, thereby delivering the reporter group into the cell. Once in the cell, the targeting nucleic acids hybridize preferentially to their specific target nucleic acid, such as an mRNA, and remain bound within the cell. Absent the targeting nucleic acid, the reporter groups are not retained within the cells.

The targeting nucleic acid can be linked to the reporter group or groups by a variety of methods, including, e.g., covalent bonds, bifunctional spacers (“bridge”) such as, avidin-biotin coupling, Gd-DOPA-dextran coupling, charge coupling, or other linkers.

The reporter groups can be contrast agents such as magnetic particles, such as superparamagnetic, ferromagnetic, or paramagnetic particles. Paramagnetic metals (e.g., transition metals such as manganese, iron, chromium, and metals of the lanthanide group such as gadolinium) alter the proton spin relaxation property of the medium around them.

There is considerable latitude in choice of particle size. For example, the particle size can be between 1 nm and 2000 nm, e.g., between 2 nm and 1000 nm (e.g., 200 or 300 nm), or between 10 nm and 100 nm, as long as they can still be internalized by the cells. The magnetic particles are typically nanoparticles. Preferably, within any particular probe preparation, particle size is controlled, with variation in particle size being limited, e.g., substantially all of the particles having a diameter in the range of about 30 nm to about 50 nm. Particle size can be determined by any of several suitable techniques, e.g., gel filtration or electron microscopy. An individual particle can consist of a single metal oxide crystal or a multiplicity of crystals.

There are two types of contrast agents useful for MR imaging: T1 and T2 agents. The presence of T1 agent, such as manganese and gadolinium, reduces the longitudinal spin-lattice relaxation time (T1) and results in localized signal enhancement in T1 weighted images. On the other hand, the presence of a strong T2 agent, such as iron, will reduce the spin-spin transverse relaxation time (T2) and results in localized signal reduction in T2 weighted images. Optimal MRI contrast can be achieved via proper administration of contrast agent dosage, designation of acquisition parameters such as repetition time (TR), echo spacing (TE) and RF pulse flip angles.

Specific examples of such magnetic nanoparticles include monocrystalline iron oxide nanoparticles (MIONs) as described, e.g., in U.S. Pat. No. 5,492,814; whitehead, U.S. Pat. No. 4,554,088; Molday, U.S. Pat. No. 4,452,773; Graman, U.S. Pat. No. 4,827,945; and Toselson et al., Bioconj. Chemistry, 10: 186-191 (1999). These particles can also be superparamagnetic iron oxide particles (SPIOs), ultra small superparamagnetic iron oxide particles (USPIOs), and cross-inked iron oxide (CLIO) particles (see, e.g., U.S. Pat. No. 5,262,176).

MIONs can consist of a central 3 nm monocrystalline magnetite-like single crystal core to which are attached an average of twelve 10 kD dextran molecules resulting in an overall size of 20 nm (e.g., as described in U.S. Pat. No. 5,492,814 and in Shen et al., “Monocrystalline iron oxide nanocompounds (ION): Physicochemical Properties,” Magnetic Resonance in Medicine, 29:599-604 (1993), to which nucleic acids can be conjugated for targeted delivery.

The dextran/Fe w/w ratio of a MION can be 1.6:1. R1=12.5 mM sec⁻¹, R2=45.1 mM sec⁻¹ (0.47 T, 38° C.). At room temperature relaxivity in an aqueous solution at room temperature and 0.47 Tesla can be: R1˜19/mM/sec, R2˜41/mM/sec. MIONs elute as a single narrow peak by high performance liquid chromatography with a dispersion index of 1.034; the median MION particle diameter (of about 21 nm as measured by laser light scattering) corresponds in size to a protein with a mass of 775 kD and contains an average of 2064 iron molecules.

The physicochemical and biological properties of the magnetic particles can be improved by crosslinking the dextran coating of magnetic nanoparticles to form CLIOs to increase blood half-life and stability of the reporter complex. The cross-linked dextran coating cages the iron oxide crystal, minimizing opsonization. Furthermore, this technology allows for slightly larger iron cores during initial synthesis, which improves the R2 relaxivity. CLIOs can be synthesized by crosslinking the dextran coating of generic iron oxide particles (e.g., as described in U.S. Pat. No. 4,492,814) with epibromohydrin to yield CLIOs as described an U.S. Pat. No. 5,262,176.

The magnetic particles can have a relaxivity on the order of 35 to 40 mM/sec, but this characteristic depends upon the sensitivity and the field strength of the MR imaging device. The relaxivities of the different reporter conjugates can be calculated as the slopes of the curves of 1/T1 and 1/T2 vs. iron concentration; T1 and T2 relaxation times are determined under the same field strength, as the results of linear fitting of signal intensities from serial acquisition: (1) inversion-recovery MR scans of incremental inversion time for T1 and (2) SE scans of a fix TR and incremental TE. Stability of the conjugates can be tested by treating them under different storage conditions (4° C., 21° C., and 37° C. for different periods of time) and performing HPLC analysis of aliquots as well as binding studies.

In some embodiments, the paramagnetic label on the probe is a metal chelate. Suitable chelating moieties include macrocyclic chelators such as 1,4,7,10-tetrazazcyclo-dodecane-N,N′,N″,N′″-tetraacetic acid (DOTA). For to be used in vivo, e.g., as MR contrast agents in a human patient, gadolinium (Gd³⁺), dysprosium (Dy³⁺), and europium are suitable. Manganese can also be used for imaging tissues other than in the brain. In other embodiments, CEST (Chemical Exchange Saturation Transfer) can be used. The CEST method uses endogenous compounds such as primary amines as reporter groups that can be linked to the ODN.

Other suitable reporter groups are labels such as near infrared molecules, e.g., indocyanine green (ICG) and Cy5.5 and quantum dots, which can be linked to the targeting nucleic acid and used in optical imaging techniques, such as diffuse optical tomography (DOT) (see, e.g., Ntziachristos et al., Proc. Natl. Acad. Sci. USA, 97:2767-2773, 2000). Fluorescent labels, such as FITCs, Texas Red, and Rhodamine can also be linked to the targeting nucleic acid. Radionuclides, such as ¹¹C, ¹³N or ¹⁵O, can be synthesized into the targeting nucleic acids to form the reporter conjugates. In addition, various known radiophannaceuticals such as radiolabeled tamoxifen (used, e.g., for breast cancer chemotherapy) and radiolabeled antibodies can be used. For example, they can be coated with dextran for attachment to the targeting nucleic acids as described herein. These radio-conjugates have application in positron emission tomography (PET). Radioisotopes, such as ³²P, ³³P, ³⁵S (short half-life isotopes) (Liu et al. (1994) Ann. Neurol., 36:566-576), radioactive iodine, and barium can also be integrated into or linked to the targeting nucleic acid to form conjugates that can be imaged using X-ray technology.

Note that two or more reporter groups, of the same or different kinds, can be linked to a single targeting nucleic acid.

The targeting nucleic acids are typically single-stranded, anti-sense oligonucleotides of 12, 15, 18, 20, 23, 25, 26, and up to 30 nucleotides in length. They are designed to hybridize to the target gene (if present in sufficient numbers in a cell), or to hybridize to a messenger RNA transcribed from the gene whose expression is to be imaged. They can be protected against degradation, e.g., by using phosphorothioate, which can be included during synthesis. In addition, by keeping the length to 30 or fewer nucleotides, the non-specific nuclease/protease response that could destroy cellular mRNA and induce a cytotoxic reaction can be avoided.

The reporter group and the targeting nucleic acid are then linked to produce the reporter conjugate using any of several known methods. For example, if the contrast agent is a MION, this molecule can be linked to a nucleic acid by phosphorothioating the oligonucleotide and labeling it with biotin at the 5′ end. The dextran coated MION can be activated and conjugated to the biotin-labeled oligonucleotide using avidin based linkers, such as NeutrAvidin® (Pierce Chem.).

In addition, liposomes, lipofectin, and lipofectamine can be used to help get the entire conjugate into a cell.

Various imaging modalities and corresponding reporter groups are reviewed and described in Min et al., Gene Therapy, 11:115-125 (2004), which is incorporated herein by reference in its entirety, including the references it cites.

### Methods of Administration

For administration, e.g., to an experimental rodent or human patient, a reporter conjugate can be diluted in a physiologically acceptable fluid such as buffered saline, dextrose or mannitol. Preferably, the solution is isotonic. Alternatively, the conjugate can be lyophilized and reconstituted before injection with a physiological fluid. The conjugate can be administered parenterally, e.g., by intravenous (IV) injection, subcutaneous injection, or intramuscular administration, depending on the tissue to be imaged. For imaging the brain, a useful route of administration is the intracerebroventricular (ICV) route. When administered intravenously, the conjugate can be administered at various rates, e.g., as rapid bolus administration or slow infusion.

When administered by IV injection and superparamagnetic iron particles are used as the paramagnetic label, useful dosages are between about 0.1 and 10.0 mg of iron per kg, e.g., between 0.2 and 5 mg/kg for a 1.5 Tesla medical scanner. As is known in this art, there is a field dependence component in determining the contrast dosage. Doses of iron higher than 10 mg/kg should be avoided because of the inability of iron to be excreted. These types of contrast agents can be used at a dosage of 0.001 to 0.1 mg/kg body weight for ICV administration in the rodents.

When administered by IV injection and chelated gadolinium is used as the paramagnetic label, the dose will be between 10 μmoles and 1000 μmoles gadolinium/kg, e.g., between 50 and 500 μmoles gadolinium/kg. Doses above 1000 μmoles/kg produce hyperosmotic solutions for injection.

The new reporter conjugates will shorten the relaxation times of tissues (T1 and/or T2) and produce brightening or darkening (contrast) of MR images of cells, depending on the tissue concentration and the pulse sequence used. In general, with highly T2 weighted pulse sequences and when iron oxides are used, darkening will result. With T1 weighted pulse sequences and when gadolinium chelates are used, brightening will result. Contrast enhancement will result from the selective uptake of the conjugate in cells that contain the target gene.

If delivered systemically, paramagnetic metal chelate-type probes will show renal elimination with uptake by the liver and spleen, and to a less degree by other tissues. Superparamagnetic iron oxide crystal-type probes are too large for elimination by glomerular filtration. Thus, most of the administered probe will be removed from the blood by the liver and spleen. Superparamagnetic iron oxides are biodegradable, so the iron eventually will be incorporated into normal body iron stores.

Various reporter groups for medical imaging are routinely administered to patients intravenously, but can also be delivered by intra-peritoneal, intravenous, or intra-arterial injection. All of these methods can deliver the new reporter conjugates throughout the body except to the brain due to the existence of the blood brain barrier (BBB). To by pass the BBB, one can either use ICV, or one can employ intrathecal injection into the Cistema Magna, or intra-arterial injection into the ascending aorta, followed by the transient breakage of BBB (e.g., via mannitol infusion). In certain situations, the BBB may be already breached because of a specific disorder, such as certain cancers.

### Methods of Imaging

MR imaging can be performed in live animals or humans using standard MR imaging equipment, e.g., clinical, wide bore, or research oriented small-bore MR imaging equipment, of various field strengths. Imaging protocols typically consist of T₁, T₂, and T₂* weighted image acquisition, T1 weighted spin echo (SE 300/12), T2 weighted SE (SE 5000/variable TE) and gradient echo (GE 500/variable TE or 500/constant TE/variable flip angles) sequences of a chosen slice orientation at different time points before and after administration of the reporter conjugate.

To determine the in vivo distribution of a particular reporter conjugate, biodistribution studies and nuclear imaging can be carried out using excised tumors of animals that have received a single dose of labeled reporter complex, e.g., MION-s-ODN. The same assay can be used to analyze the biodistribution of other new reporter conjugates.

To determine whether expression of a specific target gene, e.g., a therapeutic transgene, can be detected with a particular reporter conjugate, animals receive an infusion of the conjugate. After injection, differences in R2* maps (inverse of T2* maps) are determined after a pre-defined period of time. If significant, the reporter conjugate can be used in clinical imaging of that specific transgene. Biodistribution studies can be used to show a higher concentration of the reporter conjugate in cells expressing the target gene compared to matched cells that do not express (or over-express) the target gene in the same animal.

This image evaluation technique can also applied to other imaging modalities such as PET, X-ray, and DOT, in which radionuclides, radioisotopes, and/or fluorescent probes are detected. Such other imaging modalities, and their corresponding reporter groups, are described in Min et al. (Gene Therapy, 11:115-125 (2004)).

### Applications

The new methods and compositions have numerous practical applications. The availability of reporter conjugates to image cellular nucleic acids, e.g., to image, gene expression, is important for monitoring gene therapy where exogenous genes are introduced to ameliorate a genetic defect or to add an additional gene function to cells.

The new methods can also used to image endogenous gene expression during development and/or pathogenesis of disease. With advances in establishing transgenic mouse models, the new compositions can be used to develop an animal line that has a target gene under the control of a given promoter under study, so that promoter activity can be directly visualized.

The new methods can also be used for imaging gene expression in deep organs using MR imaging, and for imaging tumors that over-express certain target genes compared to normal cells.

Moreover, imaging of gene expression by high-resolution MR imaging will have a major impact in the treatment of CNS disease such as brain tumors or neurodegenerative diseases such as Alzheimer's. First, the new reporter conjugates can be used for in vivo monitoring of gene expression. This will have direct applications in determining efficacy and persistence of gene therapy by non-invasive imaging and imaging gene expression over time in the same subject. By combining previously developed techniques for tracking virions or other gene delivery vehicles with gene expression imaging, one would also be able to directly compare gene delivery and gene expression in vivo. This provides a powerful tool to study the mechanism by which viral and non-viral vectors accumulate in and transduce/transfect tumors.

The new methods will also be useful in testing many of the anticipated new vectors that are currently being designed in an effort to create safer and more efficient gene delivery systems. In addition, there are a number of strategies to improve viral gene delivery to brain tumors, either by modifying the blood-brain-barrier (BBB) or by targeting viruses. Irrespective of the strategy, methods that can quantitate delivery and follow gene expression over time are necessary tools in the development of gene therapy.

The new reporter conjugates and methods can also be used to image knockdown gene products that may be harmful to normal brain function We note that a change of as few as 3 nucleotides in the ODN of 26 nucleotides in length will significantly reduce binding of the ODN to the wild-type mRNA (Liu et al., Ann. Neurology, 36:566-576, 1994) in the brain of live animals. Therefore, one can alter a given ODN sequence so that its binding to a mutant mRNA transcribed from a mutant gene will remain at 100% homology, while binding to a wild-type mRNA transcribed from a normal gene is reduced. This type of targeting nucleic acid can be used to detect mutant target genes. Moreover, this reporter conjugate can also be used in cancer therapy. The mutant ODN can be synthesized to be complementary to a mutated oncogene, and can be designed to carry one or more anti-cancer agents, such as radiopharmaceutical or radioisotopes that can inhibit or kill the cancer cell (see FIGS. 14A to 14C). Furthermore, the ability of the reporter conjugate to discriminate between the mutant copy and wild-type copy of a target mRNA transcribed from an oncogene can be used to enable the reporter conjugate to preferentially bind to the mutant mRNA, thereby inhibiting translation of the mutant mRNA into a gene product, and thus inhibit expression of the mutant oncogene.

In another example, cancer cells are known to have higher abilities of endocytosis, which allows cancer cells to take up extracellular particles such the MION-s-ODN reporter conjugate. As shown in FIGS. 15A and 15B, in Fisher 344 rats bearing a ENU-induced glioblastoma, the s-ODN that was used in the reporter conjugate had a higher affinity to glioblastoma cells than to normal neurons. This preferential ability to bind to the glioblastoma cells can be used to deliver cancer therapeutics via direct or indirect linkage, with or without tumor-specific antibody, on the reporter contrast agent. Thus, the reporter conjugates shown in FIGS. 14A and 14B can be used for detection, diagnosis, and therapy, e.g., for treating cancers such as brain tumors.

In other embodiments, the new reporter conjugates can also be used to image apoptosis (programmed cell death, PCD) (See Example 12). For example, FIG. 16 shows that early PCD can be detected in CA neurons using TUNEL (terminal UTP nick end labeling) in postmortem brain tissue after global stroke in mice. PCD starts with self-destruction of nuclear DNA (DNA fragmentation is detected using the TUNEL assay), and then other components of the cell; dead neurons are removed and are generally not immediately replaced. PCD is believed to be involved in many neurological disorders, although the exact mechanism is not understood. At present, early PCD can be detected only in postmortem tissue samples. Reporter conjugates can be used to detect neurons that undergo PCD as follows.

First, we have shown that the reporter conjugate MION-s-ODN is taken up and distributed in live neurons (see, e.g., FIGS. 2, 4, and 13). Second, once individual neurons are committed for PCD, they will either fail to take up the reporter conjugate or those that take up the reporter conjugate will be metabolized and not visible in MR or other imaging modalities. Thus, disrupted MR images of CA neurons will appear as an indication that PCD has occurred, e.g., after stroke or other neurological disorder such as Alzheimer disease or Parkinson's disease.

In other embodiments, the new reporter conjugates can be used more generally for non-invasive detection of gene expression, cell mapping, gene targeting, phenotyping, and detection of gene arrays using several unique ODNs linked to different unique reporter groups (see, e.g., Example 11). The new conjugates can also be used to deliver chimeric reporter groups, e.g., two or more different reporter groups linked to the same targeting nucleic acid, to specific cells, with or without the use of antibodies that specifically bind to cell-surface antigens.

The new reporter conjugates can be used to detect expression of an oncogene, e.g., a mutant proto-oncogene or a mutant tumor suppressor, in a tumor or cancerous cell at a very early stage in tumor development. Several oncogenes and tumor suppressors, such as ras and p53, are known in the art.

In other embodiments, the new reporter conjugates can be used to detect the gene expression of stem cells. Stem cells have specific patterns of gene expression, depending on the type of stem cell (see, e.g., Pain et al., J. Biol. Chem., 280:6265-8 (2005)). Stem cells can be visualized, e.g., following implantation (e.g., before, during, or after stem cell therapy) in a subject.

In other embodiments, the new reporter conjugates can be used to detect the expression of a transgene in a subject. The new reporter conjugates can be used to localize expression of a transgene in a subject. The expression of a transgene that is expressed conditionally (e.g., from a conditional promoter) or tissue specifically (e.g., from a tissue-specific promoter) can be imaged using the new reporter conjugates.

The new reporter conjugates made with either DNA or RNA as the targeting nucleic acid can also be used to deliver any reporter molecule to any specific cellular nucleic acid, such as a gene, in a collection of cellular nucleic acids, such as a gene bank.

### EXAMPLES

The invention is further described in the following examples, which do not limit the scope of the invention described in the claims.

### Example 1

### Preparation of MION-s-ODN

A phosphorothioated ODN (s-ODN) 26-mer or 15-mer labeled with biotin on the 5′ end was used as the targeting nucleic acid portion of the reporter conjugate; the sequence of the 26-mer has been reported (CATCATGGTCGTGGTTTGGGCAAACC (SEQ ID NO:1); Liu et al., 1994, Arm. Neurol., 36:566-576) as has that of the 15-mer (GGCAAGCCATGTCTG (SEQ ID NO:2); Paramentier-Batteur et al., 2001, Cereb. Blood Flow Metab., 21:15-21). The 26-mer s-ODN binds to target c-fos mRNA and interferes with the expression of Fos/AP-1 activities after stroke of rats (Cui et al., 1999, J. Neurosci., 19:2784-2893; Zhang et al., 1999, Brain Res., 832:112-117).

The reporter group was a dextran-coated contrast agent, either a monocrystalline iron oxide nanoparticles (MION) or a ultra-small superparamagnetic iron oxide particle (USPIO), that was activated and conjugated using NeutrAvidine® (Pierce Chem,). Neutravidin-dextran-coated MION particles were covalently bound to the s-ODN to form the novel reporter conjugates. To monitor s-ODN uptake, the 3′-OH terminus of s-ODN was labeled using terminal transferase in the presence of digoxigenin (dig)-dUTP, and the resulting 5′biotin-s-ODN-3′dig was purified using a dextran column. Dig-labeled 5′biotin-s-ODN was mixed with 5′biotin-s-ODN at a molar ratio of 1:20 and stored at minus 20° C.

More specifically, functional groups were attached to MIONs or USPIOs (5 ml at 2 mg iron per ml) in the presence of 10 ml of sodium hydroxide at 3 N, mixed, and 3.48 g of chloroethylamine (final concentration of NaOH is 1.5N, chloroethylamine is 2 M in 15 ml) was added. The mixture was incubated with slow stirring at room temperature overnight in a well ventilated room. The solution was made neutral using HCl or NaOH, followed by filtration and three washings in 20 ml of 100 mM phosphate buffered saline (PBS, pH 7.4) using a membrane with cutoff at 100,000 Dalton (Millipore) to a final volume of 5 ml.

NeutrAvidin® was attached to functional groups on the dextran coating on the MIONs and USPIOs using an aldehyde-activated dextran coupling kit (Pierce, Rockford, Ill.). Briefly, 20 mg of activated MION or USPIO (5 mg/ml) was added to 10 mg NeutrAvidine® (2.5 mg/ml PBS) and the volume was adjusted using phosphate buffered saline (pH 7.4) to a final volume of 10 ml. Then, 0.9 ml of cyanoborohydride (64 mg/ml PBS) was added and incubated overnight at room temperature, followed by three washings in sodium citrate (25 mM, pH 8) using repeat filtrations in filter-membrane (100 kD cut off). The final volume was 5 ml (iron was 3-4 mg/ml). The solution is stable if it is stored in 4° C. in an amber coated and rubber-sealed bottle.

Ten μl of biotinylated phosphorothioated ODN (s-ODN at 1 μmole/ml) was added to 50 μl of neutravidin-MION, and incubated for at least 30 minutes at room temperature, followed by filtration and washing in filter-membrane (100 kD cut off) to form the complete reporter conjugate MION-s-ODN.

### Example 2

### Delivery of MION-s-ODN Conjugates

We investigated two groups of mice in this study, control animals with MION only and mice with the novel conjugate, MION-s-ODN (SEQ ID NO:1). Anesthesia was induced with ketamine (100 mg/kg, i.p.) plus xylazine (16 mg/kg, i.p.) to male C57bB6 mice (23-25 g, Taconic Farm, NY), and surgery was performed as described previously (Cui et al., 1999), except MION or MION-s-ODN was delivered to the brain via intracerebroventricular route (LR: −1.0, AP: −0.2, DV: −3.0 to the Bregma). Immediately before use, 5′biotin-s-ODN-3′dig/5′biotin-s-ODN mixture was conjugated to neutravidin-dextran-MION for 30 minutes at room temperature. A total of no more than 2 μl of artificial CSF (aCSF) containing either MION-s-ODN or MION-dextran (control) was infused over 5 minutes into the left lateral ventricle guided by a stereotaxic device. At fixed times after delivery (30 minutes, 3 hours for control, and additional 24 and 48 hours for animals that received MION-s-ODN); the animals were anesthetized, except the 30-minute time point, with pure O₂ plus 2% halothane (800 ml/min flow rate) and placed in a home-built cradle for MR scanning.

### Example 3

### MRI of Mouse Brain After the Delivery of MION-s-ODN

All scanning was done in a 9.4T MRI system (Bruker-Avance). A home built 1 cm transmit/receive surface coil was placed on the head of the animal. The MRI scanning protocol at each time point was as follows: serial multi-slice T2 weighted gradient echo (GE) (TR=500 ms, TE=2.3, 3, 4 and 6 ms, flip angle 30, 128×128 pixels, 0.5 mm slice, 20 slices, 15 mm FOV, 4 averages) were performed along the axial and sagittal planes. Image analysis was performed using MRVision® software (MRVision Co, Winchester, Mass.), MATLAB® (The MathWorks Inc., Natick, Mass.), and in-house software to construct T2* maps. In general, these acquisition sequences are readily available in any clinical MRI system. T2* maps can be calculated by the data processing software package included in the imaging system.

Regions of interest (ROI) were extracted (as indicated in the result section), in particular along the cortices of the brain, close to as well as away from the ventricle and the injection sites. T2 weighted GE images of TE 2.3 ms were compared at two time points (at less than 30 minutes, and either at 3 hours (to look for wash out) or one day (to look for retention) after infusion).

Specifically, we analyzed the five slices that included the injection track in the center slice (referenced as −0.2 mm to Bregma), along with the two anterior (referenced as −0.7 and −1.2 mm to Bregma) and two posterior (referenced as +0.3 and +0.8 mm to Bregma) slices. T2* values inversely correspond to the concentration of MION within the brain region. The orientation of presentation was from left to right, and is from the posterior to anterior part of the brain.

FIG. 7 provides a comparison of T2* maps of an animal that received MION-s-ODN (SEQ ID NO:1) at <30 minutes and one day post infusion. The expansion of reduced T2* values away from the ventricle at 1 day is indicative of MION presence.

### Example 4

### MRI of Mouse Brain after the Delivery of Non ODN-Conjugated MION

To determine the specificity of s-ODN, we replaced A26r (SEQ ID NO:1) with biotinylated dATP or dUTP to create a control conjugate. FIGS. 9A and 9B are each a series of MR images (GE, TE/TE=500/2.3 ms) of mouse brains acquired at two time points: 30 minutes and 3 hours after infusion of MION-dextran (FIG. 10A) (a control that shows no retention) or MION-dUTP (FIG. 9B)(another control that also shows no retention). When single nucleotide dUTP or dATP was linked to the MION-dextran, washout of MION was observed within three hours (FIG. 9B). Therefore, MION can be retained in, the brain and the retention is dependent on ODN labeling.

### Example 5

### Quantification of MION-s-ODN Uptake and Gene Expression in Mouse Brain

T2* values collected from each animal were compared between two time points within similar regions of the brain: less than 30 minutes after the infusion procedure and more than 24 hours after infusion. ANOVA statistical analysis was performed in the Prism Graph Pad® software packages.

FIGS. 10A to 10C are bar graphs (10A and 10B) and a series of MR images (10C). FIGS. 10A and 10B compare R2*(1/T2*) values in contralateral cortical regions (boxes in FIG. 11C) from selected brain slices of mice injected with MION-sODN (SEQ ID NO:1) and MION-dextran immediately (<30 minutes, 10A) or 1 day after infusion (10B). Due to the small size of a mouse brain and the interference image artifact (e.g., extensive region of great signal reduction) caused by the air-tissue interface in ears and trachea as well as intraventricular retention of MION, selection of the brain slices and regions of interest should be limited to areas of least artifact. The regions selected for the analysis are shown in FIG. 10C.

Immediately following infusion, there was no significant MION-retention (relaxivity in second-1) (p>0.05) in the contralateral cortices in animals that received MION-dextran and MION-s-ODN (FIG. 10A), suggesting equal delivery of MION. One day after infusion, MION-retention in the contralateral cortex to the infusion site (within one mm) was significantly higher (two-way ANOVA, p<0.01) in the animals that received MION-s-ODN than in those that received MION-dextran only (FIG. 10B). MION retention in animals that received MION-dATP was not significantly different from those that received MION-dextran.

The bar graphs show that the brain cells retained significant amounts of the MION reporter conjugate, and that it was distributed from the left ventricle (infusion site) to the right ventricle and the cortex.

### Example 6

### Postmortem Tissue Preparation

At various given times before or after MION-sODN (SEQ ID NO:1) or MION-dextran infusion, the animals were anesthetized for transcardial perfusion with 20 ml heparinized saline (2 units) at the rate of 10 ml/min, followed by 20 ml of 4% paraformaldehyde (PFA) in 0.1 M phosphate buffer (PBS), pH 7.4 at a rate of 10 ml/in. The brain was removed and kept in the same perfusate for at least 4 hours at 4° C., followed by chase and storage in PBS with 20% sucrose solution. The brain was then processed, and embedded in paraffin. Coronal tissue slices (each at 6-microns) were cut posterior or anterior to the injection site for immunohistochemical staining. Paraffin embedded tissue sections were de-waxed using xylene, chloroform, and dehydration in serial ethanol (100%, 95%, and then 75%).

### Example 7

### In Situ Immuno-Histochemistry of the Presence of s-ODN

Digoxigenin-s-ODN (SEQ ID NO:1) was stained using FITC conjugated sheep anti-dig IgG (Cui et al., 1999). The stained slides were evaluated using a fluorescence microscope to observe the presence of nuclear s-ODN. The s-ODN has been shown to be able to enter brain cells by itself when delivered via ICV route (Liu et al., 1994; Cui et al., 1999). FIGS. 4 and 13A-C show the nuclear uptake of s-ODN as clustered bright dots.

### Example 8

### Detection of Intracellular Presence of MION

The presence of iron oxide was detected using Prussian blue, followed by fast nuclear red counter staining (Fisher Chem. Co).

We observed the presence of iron oxide (blue-green color using Prussian blue staining for iron) and nuclear fast red for nuclei counter-stain (pink-red) in animal brains that received MION-s-ODN (SEQ ID NO:1; FIGS. 3A-C). Iron oxide was present in the cortex (FIG. 3B), CA neurons (FIG. 3C), and densely in the corpus callosum (FIG. 3A).

We observed what appeared to be the diffusion of MION-s-ODN from one aggregate in the cortex (FIG. 3B). Iron oxide was also present on the molecular layer and Purkinje cells. No iron oxide was present in animals that received only MION-dextran. In addition, the presence of iron oxide in tunnels connecting cerebroventricular walls (arrows, FIG. 3A) suggests that the MIONs probably gained access to, or were removed from, brain cells via tunnels connecting the cerebro-ventricular wall for access to the CSF.

### Example 9

### Specificity of Binding In Vivo

This example demonstrates that A26 (SEQ ID NO:1) binds specifically to c-fos mRNA after cellular uptake of MION-A26. Intracellular MION-A26 can specifically form a hybrid with c-fos mRNA in vivo and serve as a primer to enable reverse transcription (RT) towards the untranscribed region on the 5′-terminus for a complementary DNA (cDNA). Reverse transcription depends on the availability of c-fos mRNA and A26-priming, without addition of the conventional (dT)₁₅ as the RT primer. The resulting cDNA can facilitate a subsequent amplification of itself by polymerase chain reaction (PCR) using additional c-fos specific primers. The control for this experiment was MION conjugates of s-ODN with a randomized (Ran) sequence (GGGATCGTTCAGAGTCTA (SEQ ID NO:3); MION-Ran) (Zhang et al., J. Nucl. Med., 42:1660-9 (2001)).

Frozen brain samples were prepared as 20 μm tissue sections and stored at −80° C. All procedures were carried out in a RNase-free environment using RNase ZAP™ (Ambion, Tex.) at room temperature unless indicated specifically. Brain samples were removed from the freezer and dried in a vacuum overnight, then were sequentially treated with 4% paraformaldehyde (20 minutes), three washings in RNase-free phosphate-buffered saline (pH 7.2), dehydration in ethanol (50, 70, 95 to 100%), and then with pronase (Biomeda Corp., CA, 10 minutes, 37° C.), washed in 0.2% glycine/PBS, followed with treatment in DNase I (Invitrogen Life Technologies, CA, 0.1 U/μl, 37° C., 10 minutes) to remove all DNA that was not protected by phosphorothioate modification (verified by the absence of amplifiable nuclear hgprt gene using PCR [Liu et al., Mutat. Res., 288:229-36 (1993)]). After washing in RNase-free water and dehydration in 100% ethanol, the c-fos mRNA was reverse transcribed in the absence of added DNA primer using reverse transcriptase (Invitrogen Life Technologies) at 37° C. for 90 minutes, followed by washing and drying as before.

Brain samples were pre-heated on a hot plate at 95° C. following addition of PCR reaction mix (Tris-HCl [20 mM, pH 8.4], KCl [50 mM], 300 μM each of a pair of upstream and A18 primers, MgCl2 [1.5 mM], dNTP [20 μM], dig-dUTP [1 μM] and Taq DNA polymerase [1 U, Invitrogen LT]), the reaction chamber was immediately sealed using AmpliCover™ and clamp (Applied Biosystems, CA). The PCR primers are the upstream primer which has a sequence matching to position 151 to 168 (5′-gcaactgagaagccaaga-3′ (SEQ ID NO:4)), and the sequence of downstream primer A18 which is complementary to positions 276-294 (5′-catcatggtcgtggtttg-3′ (SEQ ID NO:5)) of the c-fos mRNA (van Straaten et al., Proc. Natl. Acad. Sci. USA, 80:3183-7 (1983)); the negative control had all substrates except the PCR primers. The samples were immediately transferred to a thermocycler (GeneAmp™ In Situ PCR system 1000, Applied Biosynthesis) preset at 55° C.

When all samples were sealed, the amplification was started after 3 minutes at 95° C. using 25 cycles of 45 seconds at 94° C., 1 minute at 55° C. and 1 minute at 68° C., followed by 10 minutes at 72° C. and soaking at 4° C. After PCR amplification, the samples were treated with Mung Bean nuclease (Promega, San Diego, Calif.) to remove the excess single-stranded primers (37° C., 10 minutes). The amplified double-stranded dig-cDNA was detected using alkaline phosphatase-anti-dig IgG and BCIP/NBT staining (Biomeda Corp, CA), or FITC-IgG against dig (Roche Applied Science, Germany) and observed directly using a mercury light source and a filter with 495 nm broad spectrum for 470 (excitation) and 525 (emission) nm wavelength). Replacement of DNase I with RNase A before reverse transcription nullified amplification.

RT-PCR amplification of c-fos mRNA was observed in brain samples infused with MION-A26 (FIGS. 17A and 17C), but no amplification was observed in the control in animals that received MION-Ran infusion (FIGS. 17B and 17D). The amplification was mostly observed in the cytoplasm surrounding the nucleus (asterisks) of the cortex (FIG. 17A). Moreover, no amplification was observed when c-fos primers were not present (FIG. 17E), or replaced with those of β-actin or Ran during PCR. The negative control contained auto-fluorescent signal that is often associated with non-neural cells located on ventricular or vascular walls (asterisks, FIG. 17E). Therefore, the PCR signal obtained was specific for c-fos mRNA.

To determine whether the specificity of MR contrast was from the targeting of c-fos mRNA by MION-A26, we compared the MR contrast by comparing R2* values in the contralateral somatosensory cortex (SSC) of animals that received MION-A26 or MION-Ran. Animals were infused with MION-Ran or MION-A26 as described above (84 pmol MION per kg). The mean R2* values in these two groups were significantly different from each other within the first day after infusion in normal animals (p<0.03, FIG. 18A). Two days after infusion, R2* values in the MION-A26 group decreased to a value that was not significantly different from those of the control group (FIG. 18A). This slight, yet significant difference may reflect the low copy number of c-fos mRNA in normal brain cells, or non-steady state of MION-A26 uptake during the first day of infusion.

### Example 10

### Distribution of MION-A26 after Cerebral Ischemia

Transcription of c-fos mRNA is elevated after global stroke or forebrain ischemia-reperfusion (FbIR), which involves transient bilateral common carotid artery occlusion (BCCAO) in C57black6 mice (Barone et al., J. Cereb. Blood Flow Metab., 13:683-92 (1993); Liu et al., J. Neurosci., 16:6795-806 (1996)). FbIR severely affects brain regions including the striatum, cortex, and hippocampus (Fujii et al., Stroke, 28:1805-10 (1997); Huang et al., Stroke, 32:741-7 (2001); Wu et al., J. Neurosci. Methods 107:101-6 (2001)), and induces no necrosis in the brain (Back et al., J. Neurol. 251:388-97 (2004)). This example demonstrates that regional uptake and retention of the MION-A26 (SEQ ID NO:1) conjugates resulted in a distribution that parallels c-fos mRNA up-regulation.

The mean R2* values were compared in two ROI from paired observations in four groups of live animals, including animals infused with MION-A26 (SEQ ID NO:1) or MION-Ran (SEQ ID NO:3), with and without 30-minute BCCAO (n=4 in each group). The signals were allowed to reach steady state over a period of two days (FIG. 18A). FIG. 18B shows that animals with BCCAO had significantly higher mean R2* values in the ROI of those animals that received MION-A26, but induced no changes when MION-A26 was replaced with MION-Ran (FIG. 18B). The result demonstrates that A26-mediated enhancement in MR contrast was positively associated with c-fos mRNA level. However, FbIR produced no increase in R2* values when using MION-Ran, providing strong evidence that the observed changes in R2* values of animals receiving MION-A26 were not an effect of elevated uptake of MION-s-ODN during cerebral ischemia.

To demonstrate the relationship between c-fos mRNA induction and the elevation of R2* values in the brains of animals infused with MION-A26 after FbIR, expression maps obtained using MR microscopy and conventional molecular assays were compared in postmortem brain samples. R2* maps obtained using a 14 Tesla MR system on the postmortem brains showed significantly augmented R2* values in the ROI after BCCAO (FbIR induction, FIG. 19B), compared to the R2* values of the brain without FbIR induction (Normal, FIG. 19A). Consistent with FIG. 18B, these R2* maps show increased R2* values occurred in both cortex and hippocampus.

Expression maps of c-fos mRNA were also obtained by in situ hybridization assay using a radioactive phosphate-labeled probe (An et al. Ann. Neurol., 33:45-7-64 (1993); Gu et al., Neurochem. Int., 30:417-26 (1997); Yang et al., Brain Res., 664:141-7 (1994)). The expression maps of c-fos mRNA were acquired using a ³³P-labeled cRNA probe, which showed a level of c-fos transcript below-detection in normal brains (FIG. 19C). The complementary RNA (cRNA) probe to target mRNA was transcribed from a cloned mouse c-fos or β-actin mRNA in the presence of ³³P-UTP followed by purification using Sephadex™ G50 column (Cui et al., J. Neurochem., 73:1164-74 (1999)). Baseline c-fos mRNA expression in normal brains was extremely low and essentially undetectable using conventional assays. To make sure that the null-signal of c-fos mRNA in those samples (FIG. 19C) was not a result from expired tissue samples, expression of β-actin mRNA transcript was detected in adjacent tissue slices in a parallel experiment using a ³³P-labeled complementary RNA probe to O-actin mRNA. Expression of β-actin mRNA was observed, suggesting that the inability to detect c-fos mRNA in the normal brain was not a result of using expired brain samples.

Using this assay, c-fos mRNA was slightly elevated within 30 minutes of reperfusion (release of BCCAO, FIG. 19D) and further elevated for another three hours (FIG. 19E). The c-fos mRNA expression map after BCCAO showed that the highest level (in descending order) of c-fos mRNA occurred in the hippocampus (most likely the dentate gyrus (DG)), the cortex (including the SSC), and hypothalamus (HT). In comparison to the corresponding MR map shown in FIG. 19B, the elevation of MR contrast in the contralateral ROI shown by MR microscopy was consistent with the c-fos mRNA maps of four reperfusion times (FIGS. 19D-19G). Elevation of R2* was observed in the cortex, hippocampus, and hypothalamus; however, increased R2* was not observed in the piriform cortex (PiC) because of pre-existing R2 susceptibility effects in the piriform cortex of the brain without BCCAO (FIG. 19A).

### Example 11

### Specific Phenotyping With Different ODN

A housekeeping gene transcript (e.g. beta actin gene, see FIG. 20B) was expressed at a higher level than c-fos mRNA (see FIG. 19C) in the brain under normal physiological condition (Cui et al., J. Neurochem., 73:1164-1174, 1999). This housekeeping gene is expressed at high level but is inert to change by experimental or surgical protocols. The MRI map in vivo also show higher R2 values for MION-s-BA25A1 (ACGCAGCTCAGTAACAGTCCGCCTA; SEQ ID NO:6) than that for c-fos mRNA (see, e.g., FIG. 20C). The beta-actin mRNA are expressed in the cortex and hypothalamus surrounding the 4^{th} ventricle (see, e.g., FIG. 20B). An ex vivo MR map using 14 Tesla system also show elevated R2* map in the cortex and the hypothalamus (see, e.g., FIG. 20A) when ODN of A26 is replaced with B 5A1 (SEQ ID NO:6). This beta actin map is different from that of the c-fos map (see e.g. FIG. 19A).

### Example 12

### Detection of Apoptotic Neuronal Death in Live Animals

Male C57black6 mice were anesthetized using ketamine (100 mg/kg, i.p.) and xylazine (16 mg/kg, i.p.). Cerebral ischemia was induced with the transient occlusion of the bilateral carotid arteries for 60 minutes, after which the occlusion was released for blood reperfusion (≧7 days). Animals were infused with the contrast probe (SEQ ID NO:1; Fe=1.6 μmoles/kg, ODN=13 nmoles/kg in 2 μl sodium citrate) via ICV route (LR:−1; AP:−0.2; DV:−3 mm, bregma). After 2 days uptake, MR images were acquired in live animals (with pure O₂ plus 2% halothane [800 ml/min flow rate]) using a 9.4 Tesla magnet. T2* maps were obtained using serial GEFI sequences (TR/TE=500/2.3, 3, 4, 6, α=30) and T2-weighted RARE sequence (TR/TE=5000/15). The animals were sacrificed after MRI for histological confirmations of neuronal death using MR microscopy in a 14T magnet (3D FLASH, TR/TE=50/18, α=20), followed by Niss1 or TUNEL (terminal UTP nick-end labeling) staining.

No evidence of necrosis was visible in T2 weighted images of the brain. FIG. 21A shows R2* values (mean and SE) in the contra-lateral hippocampus of 5 animals 7 days after a global stroke. We observed a significantly reduced level (p<0.05) of MION-ODN in the contra-lateral hippocampus after global stroke/heart attack (open bars) compared to the normal brains (filled bars). FIGS. 21B and 21C show MR images of the Comu Ammonis from the 14 Tesla magnet. The presence of MION was observed in normal animals (FIG. 21B; n=4); however, MION was absent in the stroke brain, particularly in the dentate gyrus and CA formations of the stroke brain (FIG. 21C). Niss1 and TUNEL staining showed nuclear and DNA fragmentation consistent with apoptosis. These data are consistent with the hypothesis that apoptotic neurons do not retain MION-ODN.

The absence of MION-retention in hippocampal neurons after stroke indicated (1) an inability to retain MION probe and/or (2) no intact target (mRNA transcript or genomic DNA) available for binding by the reporter probe in these apoptotic neurons. These results indicate that this contrast probe allows the MR detection of apoptosis in live animals.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of imaging a cellular nucleic acid in a tissue in vivo, the method comprising
obtaining a reporter conjugate comprising a targeting nucleic acid linked to a reporter group, wherein the targeting nucleic acid hybridizes to a target nucleic acid molecule corresponding to the cellular nucleic acid to be imaged;
administering the reporter conjugate to the tissue in an amount sufficient to provide a detectable image;
allowing sufficient time to pass to allow any unbound reporter conjugate to leave the tissue; and
imaging the tissue, wherein a detectable image of the reporter group in the tissue indicates the presence of the cellular nucleic acid.

2. The method of claim 1, wherein the target nucleic acid molecule comprises a messenger RNA transcribed from a target gene, and the targeting nucleic acid comprises an anti-sense strand that hybridizes to a portion of the messenger RNA, wherein the presence of the cellular nucleic acid indicates expression of the target gene.

3. The method of claim 2, wherein the target gene is a therapeutic gene previously delivered to the tissue.

4. The method of claim 1, wherein the tissue is brain tissue.

5. The method of claim 1, wherein the tissue is heart, lung, liver, pancreas, spinal cord, prostate, breast, gastrointestinal system, ovary, or kidney tissue.

6. The method of claim 1, wherein the reporter group is a superparamagnetic iron oxide particle whose maximum diameter is between 1 nm and 2000 nm.

7. The method of claim 1, wherein the tissue is in a human patient.

8. The method of claim 1, wherein the reporter conjugate is administered by intravenous injection.

9. The method of claim 1, wherein the reporter conjugate is administered via intra-cerebroventricular infusion.

10. A reporter conjugate for imaging a cellular nucleic acid consisting of a single targeting nucleic acid linked to one or more superparamagnetic iron oxide particles whose maximum diameter is between 1 nm and 1000 nm.

11. The reporter conjugate of claim 10, wherein the particle is a monocrystalline iron oxide nanoparticle (MION), ultra small superparamagnetic iron oxide particle (USPIO), or cross-linked iron oxide (CLIO) particle.

12. The reporter conjugate of claim 10, wherein the maximum diameter of the particle is between 10 nm and 100 nm.

13. The reporter conjugate of claim 10, further comprising cross-linked dextran surrounding the particle.

14. A method of imaging target cells that are undergoing or have undergone programmed cell death in a tissue, the method comprising
obtaining a reporter conjugate comprising a targeting nucleic acid linked to a reporter group, wherein the targeting nucleic acid hybridizes to a target nucleic acid molecule corresponding to the target cells;
administering the reporter conjugate to the tissue in an amount sufficient to provide a detectable image;
allowing sufficient time to pass to allow any unbound reporter conjugate to leave the tissue; and
imaging the tissue, wherein a presence of a detectable image of the reporter group in the tissue indicates that the cells in the tissue have not undergone programmed cell death, and an absence of a detectable image of the reporter group indicates that the cells are undergoing or have undergone programmed cell death.

15. The method of claim 14, wherein the cells are cornu ammonis (CA) neurons.

16. A method of treating a disorder in a patient, the method comprising:
obtaining a conjugate comprising a targeting nucleic acid linked to a therapeutic agent and a reporter group, wherein the targeting nucleic acid hybridizes to a target nucleic acid molecule corresponding to a target organ or tissue; and
administering the conjugate to a patient in an amount sufficient to treat the disorder.

17. The method of claim 16, wherein the disorder is a cancer.

18. The method of claim 17, wherein the targeting nucleic acid preferentially binds to an oncogene.

19. The method of claim 17, wherein the targeting nucleic acid preferentially hybridizes to a mutant mRNA transcribed by an oncogene.

20. Use of a reporter conjugate comprising a targeting nucleic acid linked to a reporter group, wherein the targeting nucleic acid hybridizes to a target nucleic acid molecule
corresponding a cellular nucleic acid, in the preparation of a pharmaceutical
composition for imaging a cellular nucleic acid in a tissue in vivo.
